(19) United States
(12) Patent Application Publication
(10) Pub. No.: US 2006/0212113 A1
(43) Pub. Date: Sep. 21, 2006

(54) Externally adjustable endovascular graft implant

(76) Inventors: Samuel M. Shaolian, Newport Beach, CA (US); Emanuel Shaoulian, Newport Beach, CA (US); Michael R. Henson, Coto de Caza, CA (US); Shahram Moaddeb, Irvine, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/361,196

(22) Filed: Feb. 23, 2006

Related U.S. Application Data
(60) Provisional application No. 60/656,073, filed on Feb. 24, 2005.

(51) Int. Cl. A61F 2/06

(52) U.S. Cl. 623/1.35

(57) ABSTRACT

A device, method, and system for treating abdominal aortic aneurysms is described, where the device is an endovascular graft implant that one or more adjustable elements. The adjustable elements provide improved performance, for example, reduced leaking. The adjustable elements are adjustable within the body of a patient in a minimally invasive or non-invasive manner such as by applying energy percutaneously or external to the patient's body. Examples of suitable types of energy include, for example, acoustic energy, radio frequency energy, light energy, and magnetic energy.

### RELATED APPLICATIONS

This application claims the benefit of U.S. Provisional Application No. 60/656,073, filed Feb. 24, 2005, the disclosure of which is incorporated by reference.

### BACKGROUND OF THE INVENTION

1. Field of the Invention

The present application relates generally to systems, methods, and devices for treating abdominal aortic aneurysms. More specifically, the present application provides an externally adjustable endovascular graft implant.

2. Description of the Related Art

An abdominal aortic aneurysm (AAA) is a bulging or ballooning out in the wall of the abdominal aorta. This large artery carries oxygen-rich blood from the heart to the lower portion of the body.

An “aneurysm” is defined as a localized dilation of an artery by at least 50% as compared with the expected normal diameter of the vessel. The term “ectasia” is used when the dilation is less than 50%. If the arteries are diffusely enlarged by 50% or more, the condition is called “arteriomegaly.” These conditions are also referred herein as “lesions.”

AAAs are referred to as “time bombs in the abdomen.” Many remain silent until they trigger a medical emergency and/or death. Because small aneurysms (about 4 cm or less) generally produce no symptoms, people may be unaware of them for years. The natural course of an untreated lesion is to expand and rupture, however. The ultimate outcome depends on how big the lesion gets, and if and when it is detected.

Over 1.5 million Americans have AAAs, most have no symptoms. But the 15,000 deaths due to this disease each year make it the 13th leading cause of death in the U.S. Men older than age 50 are at the greatest risk: AAAs are one of the major causes of death in this age group. Although AAAs also occur in women, the proportion of affected men to women is greater. Approximately 200,000 new cases are diagnosed each year. About 50,000 to 60,000 surgical AAA repairs are performed annually. The incidence of AAA increases with age, affecting from about 5% to about 7% of Americans older than age 60.

Treatment

The operative risk associated with elective surgical aneurysm repair is dramatically lower than the operative risk after rupture. Age should not determine whether elective repair of a large abdominal aortic aneurysm is performed in otherwise healthy elderly patients. Abdominal aortic aneurysms greater than about 5 cm in diameter usually should be repaired. Recen research suggests repairing AAA in women with mean diameters of about 5 cm because women tend to rupture smaller aneurysms. Repair of slightly smaller lesions may be considered, particularly if serial ultrasonograms show progressive enlargement and if the patients are other wise healthy.

Another treatment option is an endovascular procedure, which is a minimally invasive, catheter-based treatment using a stent. The stent is usually delivered on a introduced to the body through the femoral artery (near the thigh) and guided up into the aorta. The stent diverts blood flow away from the walls of the aneurysm. The success rate of this procedure has been estimated at about 90% in some studies.

Common Complications

Endovascular devices rely on radial force and/or hooks to engage the more normal segments of the aorta and iliac arteries, thereby excluding blood flow from the aneurysmal sac. If the proximal neck is too wide or too short or densely calcified, a good seal may not be achieved at the attachment site. An incomplete seal around the stent that permits blood to leak into the aneurysm is referred to as an “endoleak.” A possible consequence of an endoleak is repressurization of the aneurysm sac, which is referred to as “endotension.”Because the sac remains pressurized, the aneurysm is still at risk of rupture. Endoleak is a common complication after stent-graft implantation. Rates of leakge after endovascular repair of aortic aneurysms are from about 2.4% to about 45.5%. Leakage is classified according to the site of origin as proximal, distal, or middle graft. Proximal and/or distal endoleaks are typically caused by incomplete fixation of the stent-graft to the aortic wall, while middle graft endoleaks are caused by graft defects or retrograde blood flow through patent arteries.

### SUMMARY OF THE INVENTION

A device, method, and system for treating abdominal aortic aneurysms, where the device is an endovascular graft implant that one or more adjustable elements. The adjustable elements provide improved performance, for example, reduced leaking. The adjustable elements are adjustable within the body of a patient in a minimally invasive or non-invasive manner such as by applying energy percutaneously or external to the patient's body. Examples of suitable types of energy include, for example, acoustic energy, radio frequency energy, light energy, and magnetic energy.

Accordingly, some embodiments described herein provide an endovascular implant for treating an abdominal aortic aneurysm, the endovascular implant comprising a body comprising an expandable frame coupled to a graft member defining a lumen, The body is substantially Y-shaped, defining an aortic arm, a left iliac arm, and a right iliac arm, each arm comprises a body end and an open end, and the open end is in fluid communication with the lumen. The endovascular implant further comprises at least one adjustable element coupled to or integrated with the body and comprising a shape memory material. The at least one adjustable element has at least a first configuration and a second configuration. The first configuration and second configuration differ in at least one dimension, and the at least one adjustable element is adjustable postoperatively from the first configuration to the second configuration in response to application of energy from an energy source external to a patient's body.

In some embodiments, the shape memory material is selected from the group consisting of shape memory metals, shape memory alloys, shape memory polymers, shape memory ferromagnetic alloys, and combinations thereof. In some embodiments, the shape memory material comprises nitinol.

In some embodiments, the at least one dimension of the second configuration is greater than the at least one dimension of the first configuration. In some embodiments, the at least one dimension is a diameter. In some embodiments, the at least one dimension length.
In some embodiments, the at least one adjustable element is disposed in proximity to the open end of at least one of the aortic arm, the left iliac arm, and the right iliac arm. In some embodiments, the graft member covers at least a portion of the at least one adjustable element. Some embodiments further comprise an adjustable element disposed in proximity to the open ends of each of the other two of the aortic arm, the left iliac arm, or the right iliac arm. Some embodiments further comprise at least a second adjustable element disposed between the open end and the body end of the at least one of the aortic arm, the left iliac arm, or the right iliac arm.

In some embodiments, the frame comprises the at least one adjustable element. In some embodiments, substantially the entire frame is the at least one adjustable element.

In some embodiments, the adjustable element comprises a closed ring. In some embodiments, the closed ring comprises a one-way ratchet.

In some embodiments, the adjustable element comprises an open ring. In some embodiments, the adjustable element comprises a spiral portion.

In some embodiments, an insulating layer is disposed on at least a portion of the shape memory material. In some embodiments, portions of the shape memory material are exposed through openings in the insulating layer.

In some embodiments, an energy-absorbing material is disposed on at least a portion of the shape memory material. In some embodiments, the energy absorbing material absorbs ultrasonic energy. In some embodiments, the energy absorbing material absorbs radio frequency energy.

In some embodiments, a loop of wire is wrapped around at least a portion of the shape memory material.

Other embodiments provide an endovascular graft implant for treating an abdominal aortic aneurysm, the endovascular graft implant comprising: means for supporting a at least a part of the endovascular graft implant; means for causing blood flow to bypass the abdominal aortic aneurysm, the means for causing blood flow to bypass the abdominal aortic aneurysm being coupled to the means for supporting; and means for adjusting at least a portion of the endovascular graft implant postoperatively from a first configuration to a second configuration using an energy source external to a patient's body, wherein the first configuration and second configuration differ in at least one dimension.

Other embodiments provide a method for treating an abdominal aortic aneurysm, the method comprising: implanting an endovascular graft implant to cause blood flow substantially to bypass the abdominal aortic aneurysm; and adjusting the at least one adjustable element from the first configuration to the second configuration. The endovascular graft implant comprises an expandable frame coupled to a graft member defining a lumen. The body is substantially Y-shaped, defining an aortic arm, a left iliac arm, and a right iliac arm, each arm comprises a body end and an open end, and the open end is in fluid communication with the lumen. The endovascular implant further comprises at least one adjustable element coupled to or integrated with the body and comprising a shape memory material, The at least one adjustable element has at least a first configuration and a second configuration, the first configuration and second configuration differ in at least one dimension and the at least one adjustable element is adjustable postoperatively from the first configuration to the second configuration in response to application of energy from an energy source external to a patient's body.

In some embodiments, the implanting is performed percutaneously. In some embodiments, the implanting comprises expanding at least a portion of the endovascular graft implant using a balloon.

In some embodiments, the adjusting is performed postoperatively. In some embodiments, the adjusting is performed in steps.

In some embodiments, the adjusting comprises applying radio frequency energy to the adjustable element. In some embodiments, the adjusting comprises applying ultrasound energy to the adjustable element. In some embodiments, the adjusting comprises applying magnetic energy to the adjustable element.

In some embodiments, the at least one adjustable element is imaged contemporaneously with the adjusting.

### BRIEF DESCRIPTION OF THE DRAWINGS

Systems, methods, and devices embodying various features of the invention are described with reference to the following drawings, which are illustrative of certain preferred embodiments rather than limiting.

FIG. 1A illustrates in perspective view an embodiment of an externally adjustable endovascular implant with adjustable rings that have adjustable diameters.

FIG. 1C schematically illustrates the endovascular implant of FIG. 1A after implantation.

FIG. 1B is a graphical representation of the change in diameter of an embodiments of an adjustable element with temperature.

FIG. 2 illustrates in perspective view another embodiment of an externally adjustable endovascular implant with adjustable rings.

FIG. 3 illustrates in perspective view an embodiment of an externally adjustable endovascular implant with an adjustable arm length.

FIG. 4A illustrates in perspective view an embodiment of an externally adjustable endovascular implant with an adjustable body. FIG. 4B illustrates the implant of FIG. 4A after adjustment.

FIG. 5 illustrates in perspective view an adjustable endovascular implant in which at least a portion of an adjustable ring is covered by graft material.

FIG. 6 illustrates in perspective view an embodiment of an adjustable endovascular implant comprising two adjustable elements on a right iliac arm.

FIG. 7 illustrates in perspective view an embodiment of an adjustable endovascular implant in which the shape of substantially the entire the body is adjustable after implantation.

FIG. 8A illustrates a top view of an embodiment of an adjustable element or ring that is not closed. FIG. 8B illustrates the adjustable element of FIG. 8A after adjustment.

FIG. 9 illustrates in perspective view an embodiment of an adjustable endovascular implant comprising the adjustable element of FIG. 8A.

FIG. 10 illustrates a top view of an embodiment of an adjustable element comprising a ratchet.

FIG. 11A illustrates in perspective view an embodiment of a spiral adjustable element comprising a groove. FIGS. 11B and 11C illustrate steps in the adjustment of the adjustable element of FIG. 11A.

FIG. 12A is a cross-section of an embodiment of an adjustable element in which a shape memory material is disposed in a recess. FIG. 12B illustrates the adjustable element of FIG. 12A after adjustment.

FIG. 13A illustrates a top view of an embodiment of an adjustable element comprising an coating layer. FIG. 13B illustrates another embodiment of an adjustable element comprising an coating layer.

FIG. 14 illustrates a perspective view of an adjustable element comprising a wire wrapping.

FIGS. 15A and 15B illustrate in cross section two embodiments of adjustable elements with convoluted shape memory elements.

FIG. 17 illustrates an embodiment of a wrappable activation device.

### DETAILED DESCRIPTION OF CERTAIN PREFERRED EMBODIMENTS

Systems, methods, and devices for reducing the shortcoming of current therapies, for example, endoleaks, use adjustable endovascular graft implants that are dynamically adjusted postoperatively using external energy sources. In some embodiments, the size and shape of the adjustable endovascular graft implant is adjustable to improve physiological performance based on the individual needs of each patient. The externally adjustable implants are also disclosed, for example, in U.S. Patent Publication Nos. 2006/0015178 A1, 2005/0288779 A1, 2005/0288777 A, 2005/0288783 A1, 2005/0288781 A1, 2005/0288782 A1, 2005/0288776 A1, 2005/0288778 A1, 2005/0288780 A1; and U.S. patent application Ser. Nos. 11/111,682, 11/123,874, 11/351,788, 60/656,451, the disclosures of which are incorporated by reference in their entireties.

In some embodiments, an adjustable endovascular graft implant is implanted into the body of a patient such as a human or other animal. The adjustable endovascular graft implant is implanted through an incision or body opening either abdominally (e.g., laparotomy) or percutaneously (e.g., through a femoral artery or vein, or other arteries or veins), as known to one skilled in the art. The endovascular graft implant is attached to the proximal and distal necks of an AAA to divert blood flow from the aneurysm with reduced endoleaking. The endovascular graft implant is selected from one or more shapes described in greater detail below.

In some embodiments, the size, dimensions, and/or shape of the endovascular graft implant is adjustable postoperatively to provide an improved seal at the proximal and/or distal necks of the AAA. In some embodiments, size, dimensions, and/or shape of the endovascular graft implant is adjustable postoperatively to facilitate removal and/or repositioning. As used herein, “postoperatively” refers to a time after implanting the adjustable endovascular graft implant and closing the body opening through which the adjustable endovascular graft implant was introduced into the patient's body. For example, in some embodiments, imaging of the AAA after implantation of the endovascular graft implant indicates potential or actual endoleaking. Thus, in some embodiments, the outer diameter of one or both of the proximal or distal ends of the endovascular graft implant is adjusted to provide an improved seal. In another example, the length of the endovascular graft implant is adjustable after implantation, thereby obviating the need to stock many different sizes of the implant. In other embodiments, length and diameter are adjustable. In some embodiments, the shape of a portion of the graft implant changes after adjustment.

As used herein, “dimension” is a broad term having its ordinary and customary meaning and includes a measure from a first point to a second point along a line or arc. For example, in some embodiments, a dimension is a circumference, diameter, radius, arc length, width, height, or the like. As another example, in some embodiments, a dimension is a distance between two segments of a coil, an anteroposterior, lateral, rostral-caudal dimension, and the like.

In certain embodiments, the endovascular graft implant comprises one or more adjustable elements comprising a shape memory material that is responsive to changes in temperature and/or exposure to a magnetic field. Shape memory is the ability of a material to regain its shape after deformation. Shape memory materials include polymers, metals, metal alloys, and ferromagnetic alloys. The endovascular graft implant is adjusted in vivo by applying an energy input sufficient to activate the shape memory material, thereby inducing a change to a memorized shape. Suitable energy sources include, for example, electromagnetic energy, radio frequency (RF) energy, X-ray energy, microwave energy, ultrasonic energy such as focused ultrasound, high intensity focused ultrasound (HIFU) energy, light energy, electric field energy, magnetic field energy, combinations of the foregoing, or the like. For example, some embodiments use electromagnetic radiation in the infrared portion of the spectrum with wavelengths from about 750 nanometers to about 1600 nanometers. This type of infrared radiation is produced by means known in the art, for example, using a solid state diode laser. In certain embodiments, one or more portions of the endovascular graft implant is selectively heated using short pulses of energy, that is, energy is cycled with at least an on period and off period. In some embodiments, the energy pulses provide segmental heating thereby allowing segmental adjustment of portions of the endovascular graft implant without adjusting the entire implant, as discussed in greater detail below.

In certain embodiments, the endovascular graft implant includes an energy absorbing material to increase heating efficiency and to localize heating in the area of the shape memory material. Thus, damage to the surrounding tissue is reduced or minimized. Energy absorbing materials for light or laser activation energy are known in the art, for example, nanoshells, nanospheres, and the like, particularly where infrared laser energy is used to energize the material. Some embodiments of the nanoparticles are made from a dielectric, such as silica, coated with an ultra thin layer of a conductor, such as gold, and are selectively tuned to absorb a particular frequency of electromagnetic radiation. In some of these embodiments, the nanoparticles range in size from about 5 nanometers to about 20 nanometers. In some embodiments, the nanoparticles are suspended in a suitable material or solution, such as saline solution. In some embodiments, coatings comprising nanotubes or nanoparticles are also useful for absorbing energy from, for example, HIFU, MRI, inductive heating, or the like.

In other embodiments, thin film deposition or other coating techniques, such as sputtering, reactive sputtering, metal ion implantation, physical vapor deposition, and chemical deposition, are used to cover portions or all of the endovascular graft implant. Such coatings are either solid or microporous. When HIFU energy is used, for example, some embodiments of a microporous structure trap and direct the HIFU energy toward the shape memory material. In some embodiments, the coating improves thermal conduction and/or heat removal. In certain embodiments, the coating also enhances radio-opacity of the endovascular graft implant. Coating materials are selected from various groups of biocompatible organic or non-organic, metallic or non-metallic materials known in the art, such as titanium nitride (TiN), iridium oxide (IrOx), carbon, platinum black, titanium carbide (TiC), and other materials used for pacemaker electrodes and/or implantable pacemaker leads. Other materials discussed herein or known in the art are also be used to absorb energy in some embodiments. In some embodiments, the coating also includes a carrier, adhesive, thermally insulating, electrically insulating, and/or protective material on or in which the energy absorbing material is embedded.

In addition, or in other embodiments, fine conductive wires such as platinum coated copper, titanium, tantalum, stainless steel, gold, or the like, are wrapped one or more times around at least a portion of the shape memory material to allow focused and rapid heating of the shape memory material while reducing undesired heating of surrounding tissues. In preferred embodiments, the wire or wires form one or more loops suitable for inductive heating. In some preferred embodiments, the wire is radio-opaque, thereby permitting imaging, for example, by MRI. In some embodiments, the wire is coated, for example, with an electrical insulator and/or thermal insulator. In some preferred embodiments, the wire is secured to the shape memory material using an adhesive, which, in some embodiments, is also an electrical insulator and/or thermal insulator. In some embodiments, the diameter of the wire is from about 0.05 mm to about 0.5 mm.

In certain embodiments, the energy source is applied surgically either during implantation or at a later time. For example, in some embodiments, the shape memory material is heated during implantation of the endovascular graft implant by contacting the endovascular graft implant with a warm object. In other embodiments, the energy source is surgically applied after the endovascular graft implant has been implanted by percutaneously inserting a catheter into the patient's body and applying the energy through the catheter. For example, in some embodiments, RF energy, light energy, and/or thermal energy (e.g., from a resistive heating element) are transferred to the shape memory material through a catheter positioned on or near the shape memory material. Alternatively, in some embodiments, thermal energy is provided to the shape memory material by injecting a heated fluid through a catheter and/or circulating a heated fluid in a balloon through a catheter placed in close proximity to the shape memory material. As another example, in some embodiments, the shape memory material is coated with a photodynamic absorbing material, which is activated to heat the shape memory material when illuminated by light, for example, from a laser diode and/or directed to the coating through fiber optic and/or optical waveguide elements in a catheter. In certain such embodiments, the photodynamic absorbing material includes one or more therapeutic agents and/or drugs that are released when illuminated by the laser light.

In certain embodiments, a removable subcutaneous electrode or coil couples energy from a dedicated activation unit. In certain such embodiments, the removable subcutaneous electrode provides telemetry and power transmission between the activation unit and the endovascular graft implant. Some embodiments of the subcutaneous removable electrode allows more efficient coupling of energy to the implant with minimum or reduced power loss. In certain embodiments, the subcutaneous energy is delivered by inductive coupling.

In other embodiments, the energy source is applied in a non-invasive manner from outside the patient's body. In certain such embodiments, the external energy source is focused to provide directional heating to the shape memory material so as to reduce or minimize damage to the surrounding tissue. For example, in certain embodiments, a handheld or portable device comprising an electrically conductive coil generates an electromagnetic field that non-invasively penetrates a patient's body and induces a current in the endovascular graft implant. The current heats the endovascular graft implant and causes the shape memory material to transform to a memorized shape. In certain such embodiments, the endovascular graft implant also comprises an electrically conductive coil wrapped around or embedded in the memory shape material. The externally generated electromagnetic field induces a current in the endovascular graft implant's coil, causing it to heat and transfer thermal energy to the shape memory material.

In certain other embodiments, one or more external HIFU transducers focus ultrasound energy onto the implanted endovascular graft implant to heat the shape memory material. In certain such embodiments, the external HIFU transducer is a handheld or portable device. The terms “HIFU,” “high intensity focused ultrasound,” or “focused ultrasound” as used herein are broad terms and are used at least in their ordinary sense, and include, without limitation, acoustic energy within a wide range of intensities and/or frequencies. For example, some embodiments of HIFU include acoustic energy focused in a region, or focal zone, with an intensity and/or frequency that is considerably less than what is currently used for ablation in medical procedures. Thus, in certain such embodiments, the focused ultrasound is not destructive to the patient's cardiac tissue. In certain embodiments, HIFU includes acoustic energy within a frequency range of from about 0.5 MHz to about 30 MHz, and a power density within a range of from about 1 W/cm² to about 500 W/cm².

In certain embodiments, the endovascular graft implant comprises an ultrasound absorbing material that is rapidly and selectively heated when exposed to ultrasound energy, and that transfers thermal energy to the shape memory material. For example, in some embodiments, an adjustable element in the endovascular graft implant comprises a shape memory element coated with an ultrasound absorbing material. The ultrasound absorbing material comprises any suitable material known in the art, for example, a hydrogel material, a microporous material, nanoparticles, carbon nanotubes, combinations thereof, and the like.

In certain embodiments, a HIFU probe is used with an adaptive lens configured to compensate for heart and respiration movement. Some embodiments of the adaptive lens have multiple focal point adjustments. In certain embodiments, a HIFU probe with adaptive capabilities comprises a phased array or linear configuration. In certain embodiments, HIFU energy is synchronized with an ultrasound imaging device to allow visualization of the endovascular graft implant during HIFU activation. In addition, or in other embodiments, ultrasound imaging is used to non-invasively monitor the temperature of tissue surrounding the endovascular graft implant, for example, by using principles of speed of sound shift and changes to tissue thermal expansion.

In certain embodiments, non-invasive energy is applied to the implanted endovascular graft implant using a magnetic resonance imaging (MRI) device. In certain such embodiments, the shape memory material is activated by a magnetic field generated by the MRI device. In addition, or in other embodiments, the MRI device generates RF pulses that induce a current(s) in the endovascular graft implant, thereby heating the shape memory material. Some embodiments of the endovascular graft implant include one or more coils and/or MRI energy absorbing material components to increase the efficiency and directionality of the heating. For example, in some embodiments, at least a portion of a shape memory material is coated with an MRI energy absorbing material, which locally heats the shape memory material. In other embodiments, a composite material is formed comprising a shape memory material and an MRI energy absorbing material. Suitable energy absorbing materials for magnetic activation energy include particulates of ferromagnetic materials. Suitable energy absorbing materials for RF energy include ferrite materials as well as other materials configured to absorb RF energy at the resonant frequencies thereof. In some embodiments, the MRI energy absorbing material comprises nanoparticles and/or carbon nanotubes.

In certain embodiments, the MRI device is used to determine the size of the implanted endovascular graft implant before, during, and/or after the shape memory material is activated. In certain such embodiments, the MRI device generates RF pulses at a first frequency to heat the shape memory material and at a second frequency to image the implanted endovascular graft implant. Thus, the size of the endovascular graft implant can be measured without significant heating. In certain such embodiments, an MRI energy absorbing material heats sufficiently to activate the shape memory material when exposed to the first frequency and does not substantially heat when exposed to the second frequency. Other imaging techniques known in the art are also useful for determining the size of the implanted device including, for example, ultrasound imaging, computed tomography (CT) scanning, X-ray imaging, positron emission tomography (PET) scanning, or the like. In certain embodiments, such imaging techniques also provide sufficient energy to activate the shape memory material.

In certain embodiments, imaging and resizing of the endovascular graft implant is performed as a separate procedure at some point after the endovascular graft implant as been surgically implanted into the patient's AAA and the opening through which the endovascular graft implant was inserted has been surgically closed. In certain other embodiments, however, it is advantageous to perform the imaging after the endovascular graft implant has been implanted, but before closing the patient's catheterization incision, to check for endoleakage. If the amount of regurgitation remains excessive after the endovascular graft implant has been implanted, energy from the imaging device (or from another source as discussed herein) can be applied to the shape memory material so as to at least partially contract the endovascular graft implant and reduce regurgitation to an acceptable level. Thus, the success of the surgery can be checked and corrections can be made, if necessary, before closing the patient's chest.

In certain embodiments, activation of the shape memory material is synchronized with a physiological signal, for example, the heart beat, during an imaging procedure. For example, an imaging technique can be used to focus HIFU energy onto an endovascular graft implant in an AAA during a portion of the cardiac cycle. For example, as the heart beats, the endovascular graft implant may move in and out of this area of focused energy. To reduce damage to the surrounding tissue, in some embodiments, the patient's body is exposed to the HIFU energy only during portions of the cardiac cycle in which the HIFU energy is focused on the portion of the endovascular graft implant of interest. In certain embodiments, the energy is gated with a signal that represents the cardiac cycle such as an electrocardiogram signal. In certain such embodiments, the synchronization and gating are configured to allow delivery of energy to the shape memory materials at specific times during the cardiac cycle. For example, in some embodiments, the energy is gated so as to only expose the patient to the energy during the T wave of the electrocardiogram signal. The physiological event is monitored by any suitable means known in the art, for example, ultrasound imaging, computed tomography (CT) scanning, X-ray imaging, positron emission tomography (PET) scanning, or the like. In some embodiments, the physiological signal is monitored using other means, for example, by electrocardiogram (ECG), sphygmomanometry, plethysmography, and the like. This synchronization permits delivery of energy at a specific time and a specific location thereby reducing damage and risk of injury to surrounding tissues during the delivery of energy to the adjustable element. In some embodiments, the adjustable element and/or the entire or a portion of the graft implant is displayed, for example, on a monitor, thereby permitting interactive application of energy to the adjustable element.

As discussed above, shape memory materials include, for example, polymers, metals, metal alloys including ferromagnetic alloys, and combinations thereof. Exemplary shape memory polymers that are useful in certain embodiments of the present invention are disclosed by Langer, et al. in U.S. Pat. No. 6,720,402, issued Apr. 13, 2004, U.S. Pat. No. 6,388,043, issued May 14, 2002, and U.S. Pat. No. 6,160,084, issued Dec. 12, 2000, the disclosures of which are hereby incorporated by reference herein. In some preferred embodiments, the shape memory polymer comprises polylactic acid (PLA) and/or polyglycolic acid (PGA). Shape memory polymers respond to changes in temperature by changing into one or more permanent or memorized shapes. In certain embodiments, the shape memory polymer is heated to a temperature between about 38° C. and about 60° C. In certain other embodiments, the shape memory polymer is heated to a temperature in a range between about 40° C. and about 55° C. In certain embodiments, the shape memory polymer has a two-way shape memory effect, wherein heating the shape memory polymer changes it to a first memorized shape and cooling changes it to a second memorized shape. The shape memory polymer is cooled, for example, by inserting or circulating a cool fluid through a catheter.

In some embodiments, shape memory polymers implanted in a patient's body are heated non-invasively using, for example, external electromagnetic radiation energy sources such as infrared, near-infrared, ultraviolet, microwave, and/or visible light sources. Preferably, the light energy is selectively absorbed by the shape memory polymer compared with the surrounding tissue. Thus, damage to the tissue surrounding the shape memory polymer is reduced when the shape memory polymer is heated to change its shape. In other embodiments, the shape memory polymer comprises gas bubbles and/or bubble containing liquids such as fluorocarbons, and is heated by inducing a cavitation effect in the gas/liquid when exposed to HIFU energy. In other embodiments, the shape memory polymer is heated using electromagnetic fields, for example, by coating with an energy absorbing material that absorbs electromagnetic energy, as discussed above.

Certain metal alloys have shape memory qualities and respond to changes in temperature and/or exposure to magnetic fields. Exemplary shape memory alloys that respond to changes in temperature include titanium-nickel, copper-zinc-aluminum, copper-aluminum-nickel, iron-manganese-silicon, iron-nickel-aluminum, gold-cadmium, combinations of the foregoing, and the like. In certain embodiments, the shape memory alloy comprises a biocompatible material such as a titanium-nickel alloy.

Shape memory alloys exist in at least two distinct solid phases called martensite and austenite. The martensite phase is relatively soft and easily deformed, whereas the austenite phase is relatively stronger and less easily deformed. For example, shape memory alloys enter the austenite phase at a relatively high temperature and the martensite phase at a relatively low temperature. Shape memory alloys begin transforming to the martensite phase at a start temperature (Mₛ) and finish transforming to the martensite phase at a finish temperature (M_{f}). Similarly, such shape memory alloys begin transforming to the austenite phase at a start temperature (Aₛ) and finish transforming to the austenite phase at a finish temperature (A_{f}). Both transformations have a hysteresis. Thus, the Mₛ, temperature and the A_{f} temperature are not coincident with each other, and the M_{f} temperature and the Aₛ temperature are not coincident with each other.

In certain embodiments, the shape memory alloy is processed to form a memorized shape in the austenite phase in the form of a ring or partial ring. The shape memory alloy is then cooled below the M_{f} temperature to enter the martensite phase and deformed into a larger or smaller ring. For example, in certain embodiments, the shape memory alloy is formed into a ring or partial ring that is larger than the memorized shape, for example, at the proximal and/or distal seal. In certain such embodiments, the shape memory alloy is sufficiently malleable in the martensite phase to allow a user such as a physician to adjust the circumference of the ring in the martensite phase by hand to achieve a desired fit for a proximal and/or distal seal. After the endovascular graft implant is implanted, the circumference of the ring can be adjusted non-invasively by heating the shape memory alloy to an activation temperature (e.g., a temperature between the Aₛ temperature and the A_{f} temperature).

When the shape memory alloy is heated to a suitable temperature and transformed to the austenite phase, the alloy changes from the deformed shape to the memorized shape. Activation temperatures at which the shape memory alloy causes the shape of the endovascular graft implant to change shape can be selected and built into the endovascular graft implant such that collateral damage is reduced and/or eliminated in tissue adjacent the endovascular graft implant during the activation process. Exemplary A_{f} temperatures for suitable shape memory alloys range between about 45° C. and about 70° C. Furthermore, exemplary Mₛ temperatures range between about 10° C. and about 20° C., and exemplary M_{f} temperatures range between about −1° C. and about 15° C. The size of an adjustable portion of the endovascular graft implant can be changed all at once or incrementally in small steps at different times in order to achieve the adjustment necessary to produce the desired clinical result.

Certain shape memory alloys further include a rhombohedral phase, having a rhombohedral start temperature (Rₛ) and a rhombohedral finish temperature (R_{f}), which exists between the austenite and martensite phases. An example of such a shape memory alloy is a NiTi alloy (Nitinol), which is commercially available from Memry Corporation (Bethel, Connecticut). In certain embodiments, an exemplary Rₛ temperature range is between about 30° C. and about 50° C., and an exemplary R_{f} temperature range is between about 20° C. and about 35° C. One benefit of using a shape memory material having a rhombohedral phase is that in the rhomobohedral phase, the shape memory material experiences a partial physical distortion, as compared to the generally rigid structure of the austenite phase and the generally deformable structure of the martensite phase.

Certain shape memory alloys exhibit a ferromagnetic shape memory effect, wherein the shape memory alloy transforms from the martensite phase to the austenite phase when exposed to an external magnetic field, for example, applied using an MRI and/or another external magnetic source. The term “ferromagnetic” as used herein is a broad term and is used in its ordinary sense and includes, without limitation, any material that easily magnetizes, such as a material having atoms that orient their electron spins to conform to an external magnetic field. Ferromagnetic materials include permanent magnets, which can be magnetized through a variety of modes, and materials, such as metals, that are attracted to permanent magnets. Ferromagnetic materials also include electromagnetic materials that are capable of being activated by an electromagnetic transmitter, such as one located outside the AAA. Furthermore, some ferromagnetic materials include one or more polymer-bonded magnets, wherein magnetic particles are bound within a polymer matrix, such as a biocompatible polymer. Some embodiments of the magnetic materials comprise isotropic and/or anisotropic materials, such as for example NdFeB (neodynium iron boron), SmCo (samarium cobalt), ferrite, and/or AlNiCo (aluminum nickel cobalt) particles.

Thus, in some embodiments, an endovascular graft implant comprising a ferromagnetic shape memory alloy is implanted in a first configuration having a first shape and later changed to a second configuration having a second (e.g., memorized) shape without heating the shape memory material above the Aₛ temperature. Advantageously, nearby healthy tissue is not exposed to high temperatures that are potentially damaging to the tissue. Further, since the ferromagnetic shape memory alloy does not need to be heated in order to change the shape, is some embodiments, the size of the endovascular graft implant is adjusted more quickly and more uniformly than by heat activation.

Exemplary ferromagnetic shape memory alloys include FeC, FePd, FeMnSi, CoMn, FeCoNiTi, NiMnGa, Ni₂MnGa, CoNiAl, and the like. Embodiments of certain of these shape memory materials also change shape in response to changes in tempereture. Thus, the shape of such materials are adjustable by exposure to a magnetic field, by changing the temperature of the material, or both.

In certain embodiments, combinations of different shape memory material are used. For example, endovascular graft implants according to certain embodiments comprise a combination of shape memory polymer and shape memory alloy (e.g., NiTi). In certain such embodiments, an endovascular graft implant comprises a shape memory polymer tube and a shape memory alloy (e.g., NiTi) disposed within the tube. Such embodiments are flexible and allow the size and shape of the shape memory to be further reduced without impacting fatigue properties. In addition, or in other embodiments, shape memory polymers are used with shape memory alloys to create a bi-directional (e.g., capable of expanding and contracting) endovascular graft implant. Bi-directional endovascular graft implants can be created with a wide variety of shape memory material combinations having different characteristics.

For example, in some embodiments, an adjustment cycle is reversible thermally. Some shape memory alloys, such as NiTi or the like, respond to the application of a temperature below the nominal ambient temperature. After an adjustment cycle has been performed on an adjustable element, cooling it below the Mₛ temperature will start reversing the adjustment cooling below the M_{f} temperature finishes the transformation to the martensite se the adjustment cycle. As discussed above, certain polymers also exhibit a two-way shape memory effect and can be used to both expand and contract an adjustable element through heating and cooling processes. Cooling can be achieved, for example, by inserting a cool liquid onto or into an adjustable element through a catheter, or by cycling a cool liquid or gas through a catheter placed near the adjustable element. Exemplary temperatures for a NiTi embodiment for cooling and reversing an adjustment cycle range between approximately 20° C. and approximately 30° C.

In some embodiments, external stresses are applied to an adjustable element during cooling to reverse the adjustment. In some embodiments, one or more biasing elements are operatively coupled to the adjustable element so as to exert a circumferential reversing force thereon.

In the following description, reference is made to the accompanying drawings, which form a part hereof, and which show, by way of illustration, specific embodiments or processes in which the invention may be practiced. Where possible, the same reference numbers are used throughout the drawings to refer to the same or like components. In some instances, numerous specific details are set forth in order to provide a thorough understanding of the present disclosure. The present disclosure, however, may be practiced without the specific details or with certain alternative equivalent components and methods to those described herein. In other instances, well-known components and methods have not been described in detail so as not to unnecessarily obscure aspects of the present disclosure.

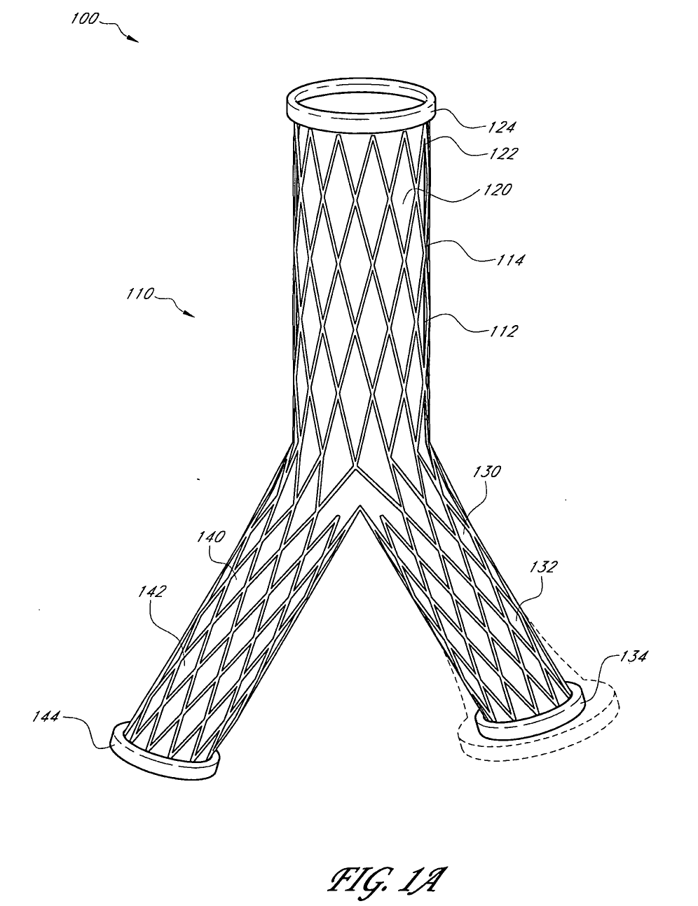

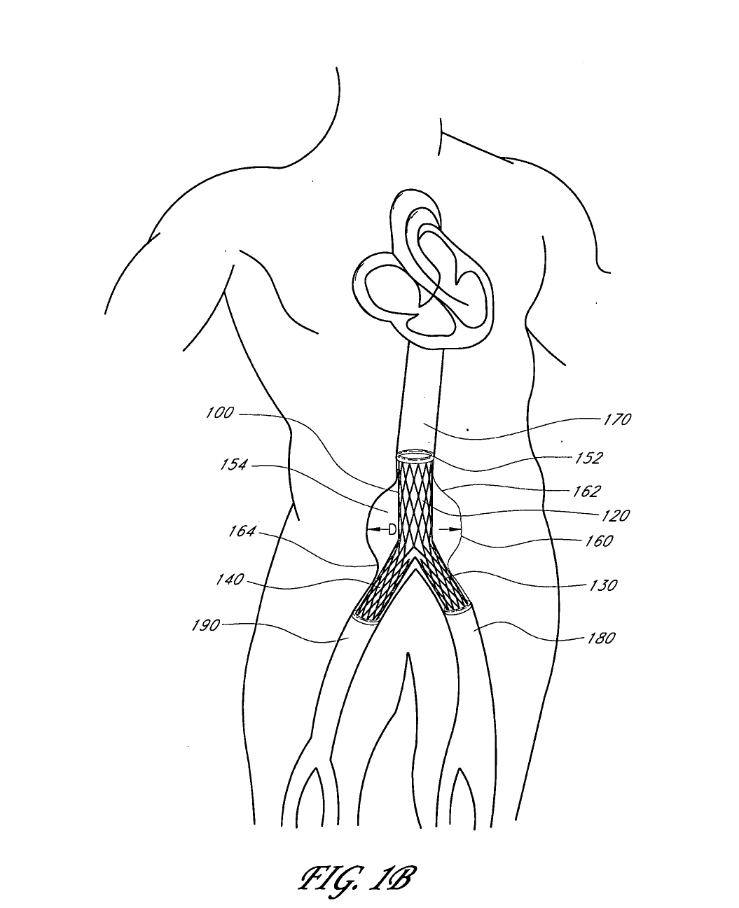

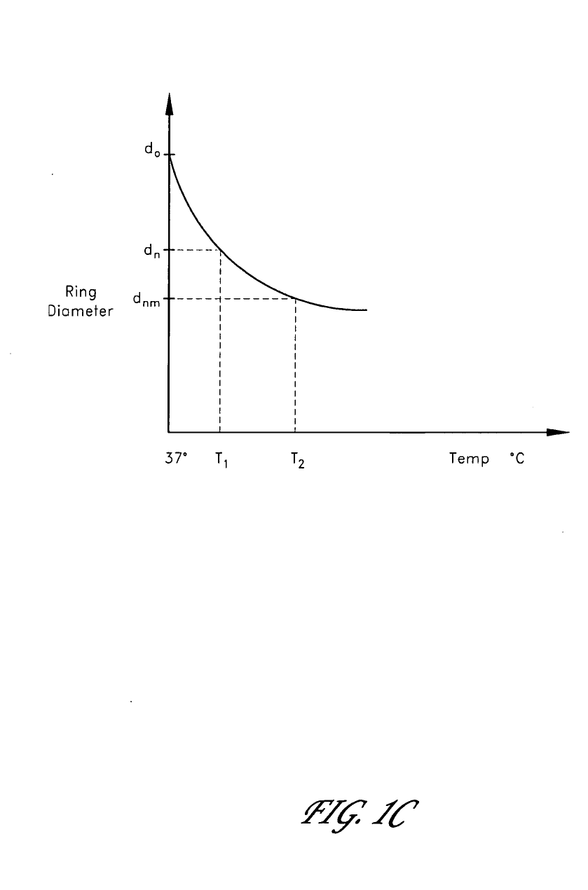

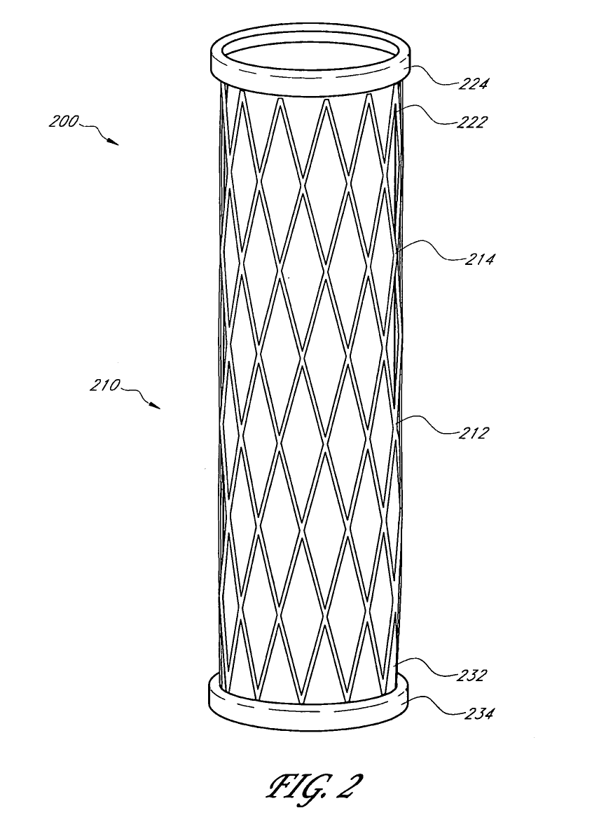

FIG. 1A illustrates an embodiment of an endovascular graft implant 100 that is adjustable after implantation. The illustrated embodiment comprises a substantially hollow, Y-shaped body 110 with an aortic arm 1.20 terminating in an open aortic end 122, a left iliac arm 130 terminating in an open left iliac end 132, and a right iliac arm 140 terminating in an open right iliac end 142. In the illustrated embodiment, the endovascular graft implant 100 is substantially symmetrical, that is, the left iliac arm 130 and right iliac arm 140 are substantially identical. In other embodiments, the graft implant 100 is not symmetrical. For example, in some embodiments, the left and right iliac arms 130 and 140 have different lengths, which is useful, for example, in implanting the implant 100 , as discussed in greater detail below. The left iliac arm 130 is illustrated in an unadjusted configuration in solid lines, and in an expanded configuration in phantom. The adjustment of the endovascular graft implant 100 is discussed in greater detail below.

In the illustrated embodiment, the body 110 comprises a graft member 112 and a frame 114. The graft member 112 defines a lumen through which blood is directed, thereby bypassing the AAA and relieving the pressure therein. The diameters of the lumens under physiological conditions in the each of the aortic arm 120, left iliac arm 130, and right iliac arm 140 will vary depending on sizes of the abdominal aorta and common iliac arteries of the patient. The frame 114 provides mechanical support to the graft implant 100, and in some embodiments, anchors the graft implant 100 to at least some degree.

In preferred embodiments, the graft member 112 comprises a graft fabric that is substantially impermeable to body fluids, for example, blood and/or plasma. The graft fabric comprises one or more biocompatible materials known in the art, for example, polyester (Dacron®), polyamide (Nylon®, Delrin®), polyimide (PI), polyetherimide (PEI), polyetherketone (PEEK), polyamide-imide (PAI), polyphenylene sulfide (PPS), polysulfone (PSU), silicone, woven velour, polyurethane, polytetrafluoroethylene (PTFE, Teflon®), expanded PTFE (ePTFE), fluoroethylene propylene (FEP), perfluoralkoxy (PFA), ethylene-tetrafluoroethylene-copolymer (ETFE, Tefzel®), ethylene-chlorotrifluoroethylene (Halar®), polychlorotrifluoroethylene (PCTFE), polychlorotrifluoroethylene (PCTE, Aclar®, Clarus®), polyvinylfluoride (PVF), polyvinylidenefluoride (PVDF, Kynar(®, Solef®), fluorinated polymers, polyethylene (PE, Spectra®), polypropylene (PP), ethylene propylene (EP), ethylene vinylacetate (EVA), polyalkenes, polyacrylates, polyvinylchloride (PVC), polyvinylidenechloride, polyether block amides (PEBAX), polyaramid (Kevlar(®), heparin-coated fabric, or the like. In some embodiments, the graft member 112 comprises reinforcing fibers known in the art, for example, fibers made from the materials discussed above, as well as fibers made from metal, steel, stainless steel, NiTi, metal alloys, carbon, boron, ceramic, polymer, glass, polymers, biopolymers, silk protein, cellulose, collagen, combinations thereof, and the like. In other embodiments, the graft member 112 comprises a biological material, for example, a homograft, a patient graft, or a cell-seeded tissue. Combinations and/or composites are also suitable.

In some preferred embodiments, the graft fabric comprises a laminate and/or composite having two or more layers. In preferred embodiments, the graft member 112 comprises a laminated graft fabric. In some embodiments, the laminate comprises one or more biologically active layers, for example, an inner and/or outer layer conducive to the proliferation of endothelial tissue, and/or that releases a drug, therapeutic agent, anti-coagulant, anti-proliferant, anti-inflammatory agent, and/or tissue growth modulating agent. In some preferred embodiments, the laminate comprises one or more mechanical and/or reinforcing layers, comprising, for example, mesh and/or fabric layers, and/or reinforcing fibers. The fabric layers are woven or non-woven. Methods for manufacturing laminated/composite fabrics are known in the art, for example, using adhesives, thermal bonding, in situ curing, and the like. Those skilled in the art will understand that such layers for useful for providing the graft member 112 with desired mechanical properties, for example, strength, elasticity, and/or the like. For example, in some embodiments, the graft fabric is elastomeric, thereby permitting the graft member 112 to expand and contract in response to blood pressure changes. In preferred embodiments, the graft member 112 in its maximally expanded state under physiological conditions is smaller than the AAA. In some embodiments, the mechanical properties of the graft member 112 are anisotropic. For example, in some embodiments, the graft member 112 is more expandable circumferentially than longitudinally.

In some embodiments, the graft member 112 has a substantially uniform thickness. In other embodiments, the graft member 112 comprises areas of different thicknesses. For example, some embodiments of a fabric laminate graft member 112 comprise extra reinforcement in areas subject to stress, for example, where the graft member 112 is likely to contact the frame 114, and/or around the ends 122, 132, and 142 of the aortic and iliac arms. In some preferred embodiments, the graft fabric is from about 0.25 mm to about 2.5 mm thick.

The frame 114 is of any suitable type known in the art. In some embodiments, the frame 114 comprises a metal, for example, titanium, steel, stainless steel, and/or, nitinol. In other embodiments, the frame 114 comprises a non-metal, for example, a polymer or ceramic. The polymer is rigid, flexible, and/or elastomeric. In still other embodiments, the frame 114 comprises a composite. In some embodiments, the frame 114 is substantially unitary. In other embodiments, the frame 114 comprises a plurality of components or subassemblies. In some embodiments, the frame 114 comprises one or more structures and/or subcomponents fabricated from wire. The term “wire” is a broad term having its. normal and customary meaning and includes, for example, mesh, flat, round, rod-shaped, or band-shaped members. In some embodiments, the frame 114 comprises one or more structures and/or subcomponents fabricated from a sheet and/or billet, for example, by stamping, drilling, cutting, forging, shearing, machining, etching, and the like. In some embodiments, the frame 114 is at least partially self-deploying. In some embodiments, a deployment device is used, for example, a balloon. In preferred embodiments, the frame 114 comprises securing means for securing the implant 100, for example, hooks, barbs, spikes, protrusions, and the like. The securing means are disposed on the frame 114 at or around the exterior of the aortic end 122 and iliac ends 132 and 142. In some embodiments, the frame 114 comprises one or more biologically active compounds and/or active chemical entities known in the art, for example, a drug, therapeutic agent, anti-coagulant, anti-proliferant, anti-inflammatory agent, and/or tissue growth modulating agent. In the illustrated embodiment, the frame 114 comprises a stent.

The illustrated embodiment 100 comprises a plurality of adjustable elements which, in the illustrated embodiment, are adjustable rings 124, 134, and 144. Those skilled in the art will understand that the following description of the adjustable rings 124, 134, and 144 is equally applicable to other types of adjustable elements. As used, the term “ring” broadly refers to shapes that are closed or open. In the illustrated embodiment, the adjustable rings 124, 134, and 144 are substantially circular, closed rings. An aortic adjustable ring 124 is disposed proximal to the aortic end 122. A left iliac adjustable ring 134 and a right iliac adjustable ring 144 are disposed proximal to the left 132 and right 142 iliac ends, respectively. In some embodiments, one or more of the adjustable rings are secured to the frame 114, to the graft member 112, or to the frame 114 and the graft member 112. Each of the adjustable rings 124, 134, and 144 is independently selected from one or more shapes, for example, a round or circular shape, an oval shape, a C-shape, a D-shape, a U-shape, an open circle shape, an open oval shape, other curvilinear shapes, and other suitable shapes. In some embodiments, the shape comprises one or more spiral portions, as discussed in greater detail below.

Each of the adjustable rings 124, 134, and 144 independently have any suitable cross-sectional shape. In preferred embodiments, the adjustable rings 124, 134, and 144 have substantially, circular, elliptical, ovoid, rectangular, trapezoidal, square, triangular, and/or hexagonal cross sections. Those skilled in the art will understand that in some embodiments, the cross sectional shape assists in the securing of one or more of the adjustable rings 124, 134, and 144 to the body 110, as discussed above. In some embodiments, one or more of the adjustable rings 124, 134, and 144 comprises means for securing the implant 100 in the body, for example, hooks, barbs, spikes, protrusions, and the like.

The outer diameter of the adjustable rings 124, 134, and 144 is expandable and/or contractible. In some embodiments, another dimension of the adjustable rings 124, 134, and/or 144 is also adjustable, for example, the length. In some embodiments, the dimensional change(s) are substantially isotropic, while in other embodiments, the changes are anisotropic. For example, in some embodiments, a substantially circular adjustable ring is substantially elliptical after adjustment.

The adjustable rings 124, 134 and 144 independently comprise one or more of the shape memory materials discussed herein, for example, metals, alloys, polymers, and/or ferromagnetic alloys. In some embodiments, one or more of the adjustable rings 124, 134, and/or 144 comprises a shape memory material that responds to the application of temperature that differs from a nominal ambient temperature, for example, the nominal body temperature of 37° C. for humans. In some preferred embodiments, the shape memory material is nitinol. Heating the adjustable ring above the A, of the shape memory material induces the adjustable ring to return to the memorized shape.

In some preferred embodiments, the adjustable rings 124, 134, and 144 are expandable. In some embodiments, the unadjusted configuration, the aortic adjustable ring 124 has on outer diameter of from about 0.5 cm to about 1.5 cm. In some embodiments, the adjusted configuration, the aortic adjustable ring has on outer diameter of from about 1 cm to about 2 cm. In their unadjusted configurations, the left 134 and/or right 144 iliac adjustable rings have outer diameters of from about 0.25 cm to about 0.5 cm. In some embodiments, the left 134 and the right 144 iliac adjustable rings have outer diameters of from about 0.5 cm to about 1 cm. In some embodiments, the expansion percentages for the adjustable rings 124, 134, and 144 is from about 6% to about 23%, where the expansion percentage is the difference between the starting and finishing diameter of the adjustable ring divided by the starting diameter. Those skilled in the art will understand that different sized adjustable rings 124, 134, and 144 are useful for different patients, for example, smaller than 0.25 cm or larger than 1.5 cm.

The activation temperatures (e.g., temperatures ranging from the Aₛ temperature to the A_{f} temperature) at which an adjustable element expands to an increased circumference is selected and built into an adjustable element such that collateral damage is reduced or eliminated in tissue adjacent the adjustable element during the activation process. Exemplary At temperatures for the shape memory material of an adjustable element at which substantially maximum expansion occurs are in a range between about 38° C. and about 1310° C. In some embodiments, the Ar temperature is in a range between about 39° C. and about 75° C. For some embodiments that include shape memory polymers for an adjustable element, activation temperatures at which the glass transition of the material or substantially maximum contraction occur range between about 38° C. and about 60° C. In other such embodiments, the activation temperature is in a range between about 40° C. and about 59° C.

In some embodiments, the austenite start temperature Aₛ is in a range between about 33° C. and about 43° C., the austenite finish temperature A_{f} is in a range between about 45° C. and about 55° C., the martensite start temperature M, is less than about 30° C., and the martensite finish temperature M_{f} is greater than about 20° C. In other embodiments, the austenite finish temperature A_{f} is, in a range between about 48.75° C. and about 51.25° C. Other embodiments can include other start and finish temperatures for martensite, rhombohedral and austenite phases as described herein.

In some embodiments, an adjustable element is shape set in the austenite phase to a remembered configuration during its manufacturing such that the remembered configuration has a relatively larger diameter. After cooling the adjustable element below the M_{f} temperature, it is mechanically deformed to a relatively smaller diameter to achieve a desired starting nominal diameter. In some embodiments, the adjustable element is sufficiently malleable in the martensite phase to allow a user, such as a physician, to manually adjust the circumferential value to achieve a desired fit the aorta or a common iliac artery.

In some embodiments, one or more of the adjustable rings 124, 134, and 144 comprises a plurality of components. For example, in some embodiments, an adjustable ring 124, 134, and/or 144 comprises a body and a means for securing the ring to the body 110, for example, screws, pins, a lock ring, a snap ring, latches, detents, springs, clips, combinations thereof, and the like. In some embodiments, one or more of the adjustable rings 124, 134, and/or 144 comprise a plurality of shape memory materials, each of which is adjustable under different conditions. For example, in some embodiments, an adjustable element comprises a plurality of shape memory materials with different A_{f} temperatures, thereby permitting a stepwise and/or sequential adjustment of the adjustable element using selective heating and/or cooling, as discussed below. In other embodiments, an adjustable element comprises two or more shape memory materials that adjust by different mechanisms, for example, a thermal shape memory material and a ferromagnetic shape memory material.

In the illustrated embodiment, the adjustable rings 124, 134, and 144 are secured to both the graft member 112 and the frame 114 by means known in the art, for example, by suturing, adhesively, mechanically, manufacturing integrally into the body 110, thermal welding and/or bonding, or combinations thereof. Examples of suitable adhesives are known in the art, and include polyurethane, polyurea, epoxide, synthetic rubbers, silicone, and mixtures, blends, and copolymers thereof. The adhesive(s) are UV curing, thermally curing, thermoplastic, and/or thermosetting. Suitable mechanical securing means include lock rings, snap rings, pins, screws, latches, detents, springs, clips, swaging, heat shrinking, and the like. Thermal welding or bonding is performed with or without an intermediate bonding layer, for example, a thermoplastic bonding film (e.g., polyethylene, polychlorotrifluoroethylene, and/or fluoroethylene propylene). In some embodiments, at least one of the adjustable rings 124, 134, and 144 is integral with at least a portion of the frame 114, for example, formed in the same manufacturing step. In some embodiments, at least one of the adjustable rings 124, 134, and 144 is secured to at least a portion of the frame 114 as discussed above.

In some embodiments, at least one of the adjustable rings 124, 134, and 144 comprises a porous structure and/or a fabric, which provides a point of attachment for the graft material and/or frame material. In some embodiments, the porous structure is useful for drug delivery, as discussed below. In some embodiments, the at least a portion of one of the adjustable rings 124, 134, and 144 comprises one or more biologically active compounds and/or active chemical entities known in the art, for example, a drug, therapeutic agent, anti-coagulant, anti-proliferant, anti-inflammatory agent, and/or tissue growth modulating agent. In some embodiments, at least a portion of one of the adjustable rings 124, 134, and 144 is covered and/or coated with a biodegradable/biocompatible material known in the art, for example. polylactic acid (PLA). In some embodiments, this coating facilitates removal.

In the illustrated embodiment, the graft member 112 is secured to adjustable rings 124, 134, and 144 as discussed above. In some embodiments, the graft member 112 also secured to the frame 114 by means known in the art, for example, using sutures, adhesives, mechanically, thermal welding and/or bonding, or combinations thereof. These methods are described in greater detail above. In some embodiments, the graft member 112 is secured to the frame 114 at or near the end of the aortic arm 122 and/or the ends of the iliac arms 132 and 142. In some embodiments, the graft member 112 is secured to the frame 114 at one or more locations on the body 110 distal to the ends of the aortic and iliac arms 122, 132, and/or 142. In some embodiments, securing the graft member 112 to the frame 114 provides one or more advantages, for example, improved durability or strength, and/or increased lumen size, which provides improved blood flow.

The endovascular graft implant 100 is dimensioned to permit implantation, for example, percutaneously through the femoral artery. In some of these embodiments, the graft implant 100 is loaded in an introduction or deployment catheter in a collapsed configuration (not illustrated), the catheter inserted into the femoral artery percutaneously, the catheter advanced to the AAA, the endovascular graft implant 100 deployed from the catheter, the endovascular graft implant 100 implanted, for example, using a balloon, and the introduction catheter and balloon removed. In some embodiments, the diameters of one or more of the adjustable rings 124, 134, and/or 144 are adjusted during implantation, for example, using a balloon and/or other means known in the art.

In the embodiment of the graft implant 100 illustrated in FIG. 1A, the left iliac arm 130 is shorter than the right iliac arm 140, which, in some embodiments, is helpful in positioning the graft implant 100 during implantation, for example, in cases in which the introduction catheter is inserted in the right femoral artery. Turning to FIG. 1B, the catheter is positioned to the AAA 160 and the graft implant 100 is partially deployed such that the left iliac arm 130 is deployed, but the right iliac arm 140 remains in the catheter. The catheter is then “backed up” such that the left iliac arm 130 enters the left common iliac artery 180. The right iliac arm 140 is then deployed and remains in the right common iliac artery 190.

In some embodiments, the graft implant 100 is adjusted in vivo by applying an energy source, for example, radio frequency energy, X-ray energy, microwave energy, ultrasonic energy such as high intensity focused ultrasound (HIFU) energy, light energy, electric field energy, magnetic field energy, combinations of the foregoing, or the .like. Application of energy sources is discussed in greater detail above. In some preferred embodiments, the energy source is applied in a non-invasive manner from outside the body. For example, as discussed above, an MRI device is useful for applying an amount of a magnetic field and/or RF pulse energy sufficient to adjust the graft implant 100. In other embodiments, the energy source is applied internally, for example, by surgically inserting a catheter into the body and applying energy through the catheter.

In some embodiments, the adjustment is performed in a single step. In other embodiments, the adjustment is performed in a plurality of steps. In some preferred embodiments, the adjustment steps are remote in time, which is useful, for example, where the AAA enlarges after initial implantation of the graft implant 100. Those skilled in the art will understand that in some preferred embodiments, different portions of the graft implant 100 are adjusted to different extents, and/or, not at all. For example, in some embodiments, each of the adjustable rings 124, 134, and/or 144 is independently adjusted.

The adjustment process, either non-invasive or using a catheter, is performed either all at once or incrementally in steps to achieve the desired amount of adjustment for producing the desired clinical result. If heating energy is applied such that the temperature of the adjustable element does not reach the A_{f} temperature for a substantially maximum shape change, partial shape memory transformation occurs. FIG. 1C graphically illustrates the relationship between the temperature of an embodiment of a contractable adjustable element and its diameter or transverse dimension. At body temperature of approximately 37° C., the diameter of the adjustable element has a first diameter do. The shape memory material is then increased to a first temperature To. In response, the diameter of the adjustable element reduces to a second diameter dₙ. The diameter of the adjustable element is then further reduced to a third diameter dₙₘ by raising the temperature to a second temperature T₂.

As graphically illustrated in FIG. 1C, in some embodiments, the change in diameter from d₀ to dₙₘ is substantially continuous as the temperature is increased from body temperature to T₂. For example, in some embodiments, a magnetic field of about 2.5 Tesla to about 3.0 Tesla is used to raise the temperature of the adjustable element above the A_{f} temperature to complete the austenite phase transition and to return the adjustable element to the remembered configuration. In some embodiments, however, a lower magnetic field (e.g., 0.5 Tesla) is initially applied and increased (e.g., in 0.5 Tesla increments) until the desired level of heating and desired contraction of the adjustable element is achieved. In other embodiments, the adjustable element comprises a plurality of shape memory materials with different activation temperatures and the diameter of the adjustable element is reduced in steps as the temperature increases.

Whether the shape change is continuous or stepwise, the diameter or transverse dimension, or another dimension of the adjustable element is assessed and/or monitored in some embodiments during the adjustment process by MRI imaging, ultrasound imaging, computed tomography (CT), X-ray, or the like. In some embodiments, where magnetic energy is being used to activate an adjustable element, for example, MRI imaging is performed at a field strength that is lower than that required for activation of the adjustable element.

FIG. 1B schematically illustrates a section of a patient with an abdominal aortic aneurysm (AAA) 160 located on the abdominal aorta 170 above the left 180 and right 190 common iliac arteries. The AAA comprises a proximal neck 162 and a distal neck 164. The endovascular graft implant 100 is implanted such that the aortic arm 120 extends into the abdominal aorta 170 above the proximal neck 162 of the AAA, and the iliac arms 130 and 140 extend below the distal neck 164 of the AAA into the left and right common iliac arteries 180 and 190, respectively. Consequently, the endovascular graft implant 100 causes blood flow to bypass the AAA 160.

The lengths or extent of an AAA from the proximal neck 162 to the distal neck 164 varies and can extend from the renal arteries to the common iliac arteries. Accordingly, some embodiments provide a series of the graft implant with different lengths, for example from about 10 cm to about 30 cm long. The maximum diameter “D” of the AAA is also indicated, and is typically from about 5 cm to about 8 cm.

Also illustrated in FIG. 1B are the locations where four different types of endoleaks occur, which as described above, permit blood to flow into the AAA. Endoleak is also referred to as perigraft flow. This classification was described in White et al. “Endoleak Classification” J Endovasc. Surg. 1998, 5.305-309. In type I, attachment leak, blood leaks where graft implant 100 seals 152 to the aorta 170 as a result of incomplete fixation of the graft implant 100 to the aortic wall. In type II, branch flow, blood enters the space 154 between the graft implant 100 and the AAA 160 through patent arteries. In type III, defect in graft or modular disconnection, blood leaks through a defect or damaged portion of the graft implant 100 into the space 154. In type IV, fabric porosity, blood leaks through the fabric of the graft member 112 into the space 154. Enlarging one or more of the adjustable rings 124, 134, and 144 in the graft implant 100 (FIG. 1A) improves the seal between the graft implant 100 and aorta 170 and or common iliac arteries 180 and/or 190, thereby reducing the incidence of type I leaks.

In some embodiments, one or more components and/or portions thereof of the graft implant 100 comprises a low friction coating, which facilitates insertion and placement of the device. For example, in some embodiments, a low friction coating is applied to at least a portion of the aortic adjustable ring 124, the left iliac adjustable ring 134, the right iliac adjustable ring 144, the graft member 112, the frame 114, or combinations thereof. The low friction coating comprises any suitable low friction coating known in the art, for example, fluorinated polymers, including EPTFE, PTFE (Teflon®), and the like. Other low friction coatings comprise lubricants known in the art, oils, and in particular non-toxic oils. In some embodiments, the low friction coating assists in removal of the device 100, if needed.

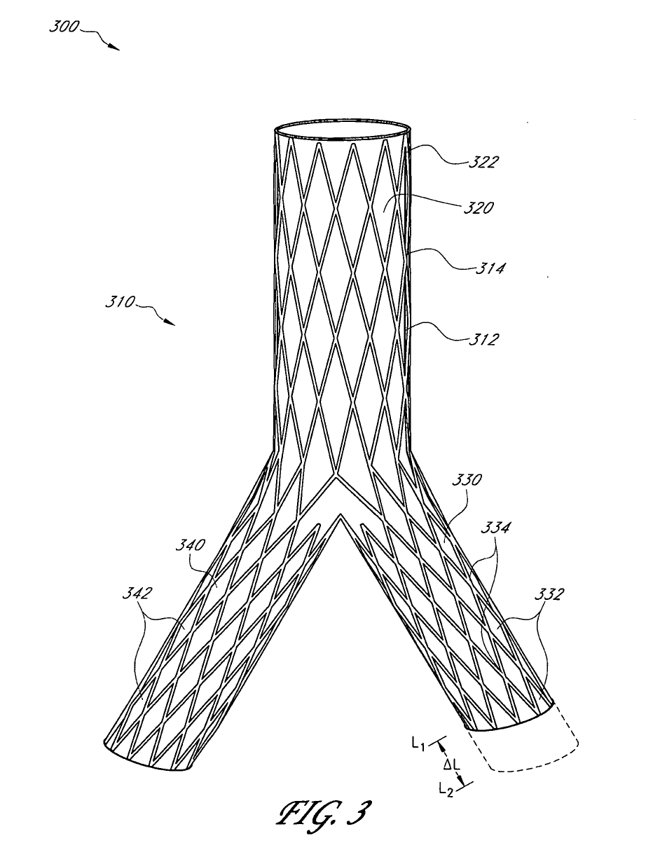

Another embodiment of the adjustable endovascular graft implant 200 is illustrated in FIG. 2, which is similar to the embodiment illustrated in FIG. 1A. The illustrated embodiment comprises a tubular body 210, comprising a graft member 212 and frame 214. The body 210 comprises a proximal end 222 and a distal end 232. The graft implant 200 comprises a proximal adjustable ring 224 and a distal adjustable ring 234. In some embodiments, the graft implant 200 is substantially symmetrical, such that the proximal and distal ends 222 and 232 ends are substantially identical. The details of the construction, materials, and the like of the illustrated embodiment are as described above for the embodiment illustrated in FIG. 1A. The embodiment illustrated in FIG. 2 is useful where the distal neck 164 of the AAA (FIG. 1B) is sufficiently distant to the common iliac arteries to permit implantation of the graft implant 100 wholly within the aorta 170. Such a structure is present in about 2-5% of patients.

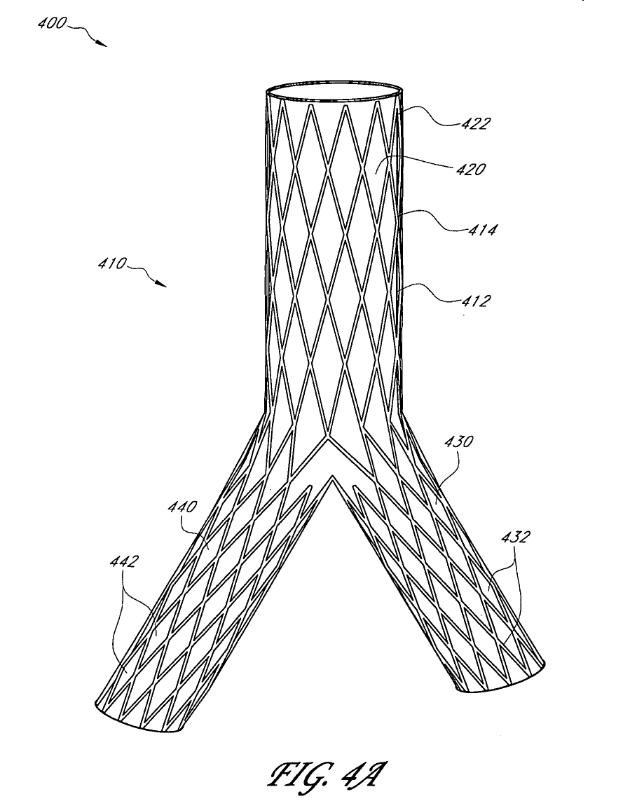

FIG. 3 illustrates an embodiment of a graft implant 300, which is similar to the embodiment illustrated in FIG. 1A. The graft implant 300 comprises a generally Y-shaped body 310, which comprises a graft member 312 and a frame 314. The body 310 comprises an aortic arm 320, a left iliac arm 330, and a right iliac arm 340. The aortic arm 320 terminates in an open aortic arm end 322. Similarly, the left and right iliac arms 330 and 340 terminate in open iliac arm ends, 332 and 342. Details of the construction and materials used in the graft implant 300 are similar to those described above for the embodiment 100 illustrated in FIG. 1A.

In the illustrated embodiment, the length of the left arm 330 is adjustable to a longer length. In some embodiments, any combination of the aortic arm 320, the left iliac arm 330, and/or the right iliac arms 340 comprise length adjustable elements. In some embodiments, each of the adjustable elements is independently adjustable. In the illustrated embodiment, the adjustable element comprises a portion of the frame 334 on the left iliac arm 330. The normal length of the left iliac arm 330 is illustrated in solid lines in FIG. 3. In phantom, the left iliac arm 330 is illustrated in an extended or lengthened configuration after activation the adjusted links. The length of the left iliac arm 330 prior to activation is indicated in FIG. 3 as length Lo. The length after activation is indicated as length L₂, which is greater than L₁. In some embodiments, activation of the adjustable material in the left iliac arm provides a decrease in length: that is, L₂ is less than L₁ as shown in the inset FIG. 3A. This length change is effected using any shape memory material described above, for example, nitinol. In some embodiments, the change in length (|L₁|-L₂|/L₁) is from about 5% to about 25%. In some embodiments, the diameter of the adjustable portion 334 also changes, for example, increases, on adjustment.

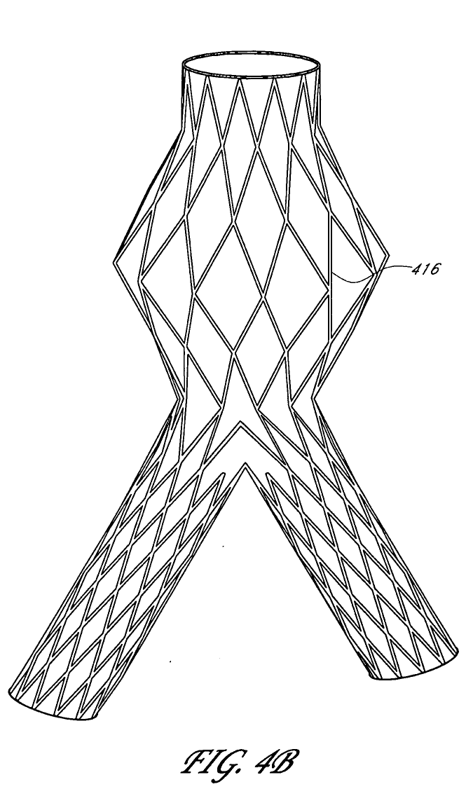

FIG. 4A illustrates another embodiment of the graft implant 400 that is similar to the embodiment illustrated in FIG. 1A. The graft implant 400 comprises a generally Y-shaped body 410 comprising a graft member 412 and a frame 414. The body 410 comprises an aortic arm 420 terminating in an open aortic arm end 422, a left iliac arm 430 terminating in an open left iliac arm end 432, and a right iliac arm 440 terminating in an open right iliac arm 442. The construction and materials for this embodiment are substantially similar as described above for the embodiment illustrated in FIG. 1A.

In the illustrated embodiment, the body 410 comprises one or more adjustable elements 416, which permit the shape of the body 410 to be adjusted post implantation. In the illustrated embodiment, the adjustable elements 416 are disposed at the base of the aortic arm 420. Those skilled in the art will understand that other configurations are possible. The adjustable elements 416 are, for example, shaped memory materials in the form of rings, wires, bands, strips, and the like. In the illustrated embodiment, the adjustable elements 416 are integrated with the frame 414. In other embodiments, the adjustable elements 416 are separate from the frame 414.

FIG. 4B illustrates the graft implant 400 after activation. In the illustrated embodiment, expanding the adjustable elements 416 causes the shape of the graft implant 400 to more closely conform to the shape of the AAA. In some embodiments, the maximum diameter of the adjusted portion is from about 5 cm to about 8 cm.

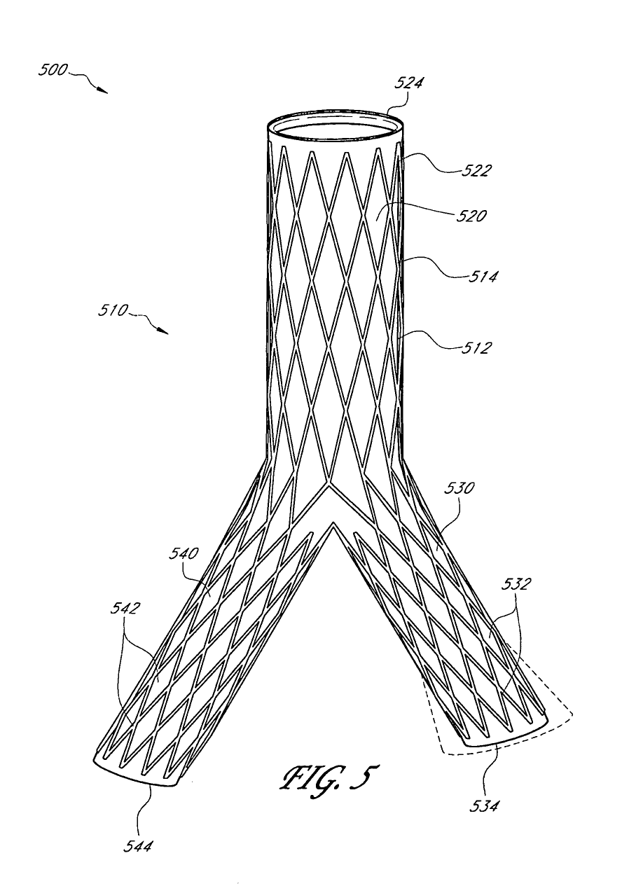

FIG. 5 illustrates another embodiment of an endovascular graft implant 500, similar to the graft implant illustrated in FIG. 1A, comprising a body 510 that comprises a graft member 512 and a frame 514. The graft implant 500 is generally Y-shaped, and comprises an aortic arm 520 terminating in an open aortic arm end 522. An aortic adjustable ring 524 is disposed proximal to the aortic end 522. The body 510 further comprises a left iliac arm 530 terminating in a left iliac arm end 532. A left iliac adjustable ring 534 is disposed proximal to the left iliac end-532. The body 510 further comprises a right iliac arm 540 terminating in a right iliac arm end 542. A right iliac adjustable ring 544 is disposed proximal to the right iliac end 542. The construction and materials of the illustrated embodiment 500 are similar as described above for the embodiment illustrated in FIG. 1A. The illustrated embodiment 500 differs from that illustrated in FIG. 1A in that at least a portion of at least one of the adjustable rings 524, 534, and/or 544 is covered with at least a portion of the graft material 512. The shape of the left iliac arm 530 before activation of the adjustable ring 534 is illustrated in solid, while the shape after activation is illustrated in phantom.

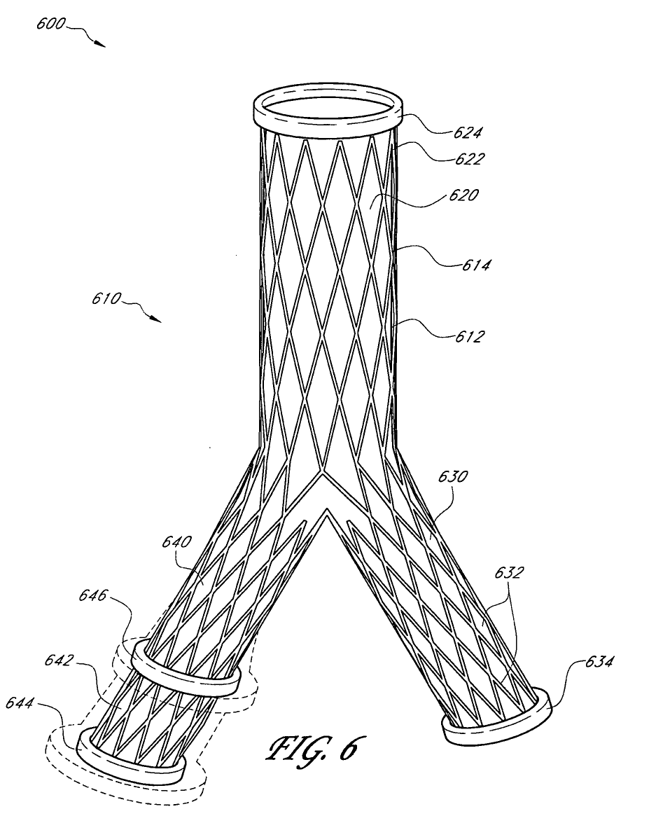

FIG. 6 illustrates an embodiment of a graft implant 600 that is similar to the embodiment illustrated in FIG. 1A. The graft implant 600 comprises a generally Y-shaped body 610 comprising a graft member 612 and a frame 614. The body 610 comprises an aortic arm 620 terminating in an open aortic arm end 622. An aortic adjustable ring 624 is disposed proximal to the aortic arm end 622. The body 610 also comprises left and right iliac arms 630 and 640, respectively, each comprising right and left iliac arm ends 632 and 642, respectively. A left iliac adjustable ring 634 is disposed proximal to the left iliac arm end 632. A first right iliac adjustable ring 644 is disposed proximal to the right iliac arm end 642, and a second right iliac adjustable ring 646 between the base of the right iliac arm 640 and the right iliac arm end 642. In the illustrated embodiment, the right iliac arm adjustable ring 644 is selectively adjustable and/or activatable to provide the structure illustrated in phantom in FIG. 6. In the illustrated embodiment, activating the adjustable ring 644 induces expansion. In other embodiments, activating the adjustable ring 644 induces contraction. Other embodiments comprise second adjustable rings on some combination of the aortic arm 620, the left iliac arm 630, and the right iliac arm 640. Other embodiments provide additional adjustable rings (more than two) on one or more of the aortic arm 620, the left iliac arm 630, and the right iliac arm 640, which provide a more fine-grained adjustability of the graft implant. Other embodiments include an additional adjustable element 648 comprising a portion of the frame between the first and second adjustable rings 644 and 646, which provides for adjustment of length and/or diameter.

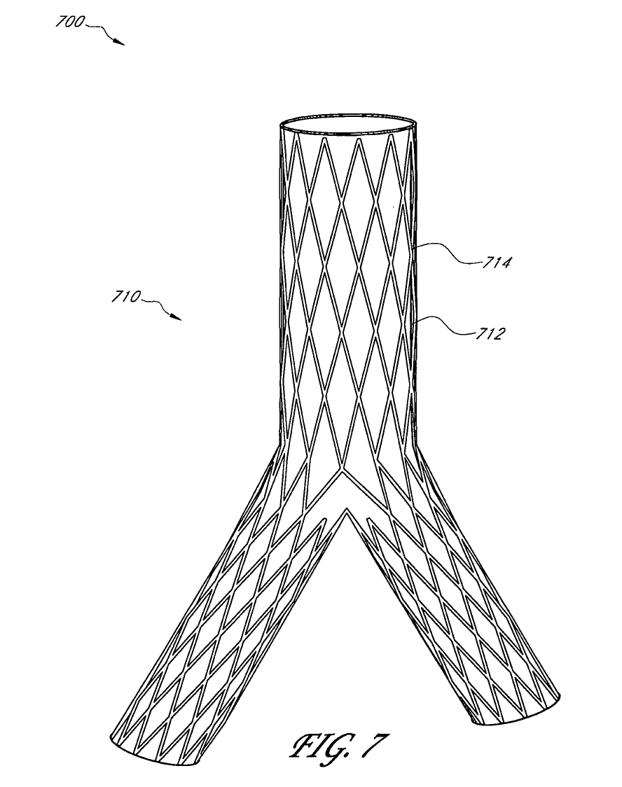

FIG. 7 illustrates another embodiment of a graft implant 700 that is similar to the embodiment illustrated in FIG. 1A. The graft implant 700 comprises a body 710 comprising a graft member 712 and a frame 714. In this embodiment, the frame 714 comprises one or more adjustable elements such that substantially the entire frame 714 is adjustable. The adjustability is expansion and/or contraction, as discussed above. For example, in some embodiments, substantially the entire frame 714 is made from a shape memory material, for example, nitinol. In other embodiments, selected components of the frame 714 are made from a shape memory material. In some of these embodiments, substantially the entire graft implant 700 is adjustable. In other embodiments, at least a portion of the implant 700 is adjustable. In some embodiments, certain portions of the graft implant 700 are adjustable to a different extent than others, for example, some portions expand more than others. In other embodiments, some portions are expandable, while other portions are contractible. For example, in some embodiments, an expandable and contractible implant 700 has two activation temperatures. At a first activation temperature, at least a portion of the implant 700 contracts, and at a second activation temperature, at least a portion of the implant 700 expands. In some embodiments, different portions of the frame 714 comprise adjustable elements with different A_{f} temperatures, thereby permitting selective and stepwise adjustment. In some embodiments, different portions of the frame 714 comprise adjustable elements with energy absorbing and/or thermally insulating coatings, and or fine wires, as discussed above, which permit selective and stepwise adjustment.

Those skilled in the art will understand that the features described for the embodiments illustrated in FIGS. 1-7 are usable in combination with each other. For example, some embodiments comprise adjustable rings as illustrated in FIGS. 1A, 2, 5, and/or 6, as well as adjustable body elements as illustrated in FIGS. 3, 4A, 4B, and/or 7. Those skilled in the art will further understand that the following exemplary and non-limiting embodiments of adjustable elements are applicable to the embodiments of the graft implant illustrated in FIGS. 1-7, as well as other embodiments not illustrated herein. Combinations of these embodiments are also within the scope of the disclosure.

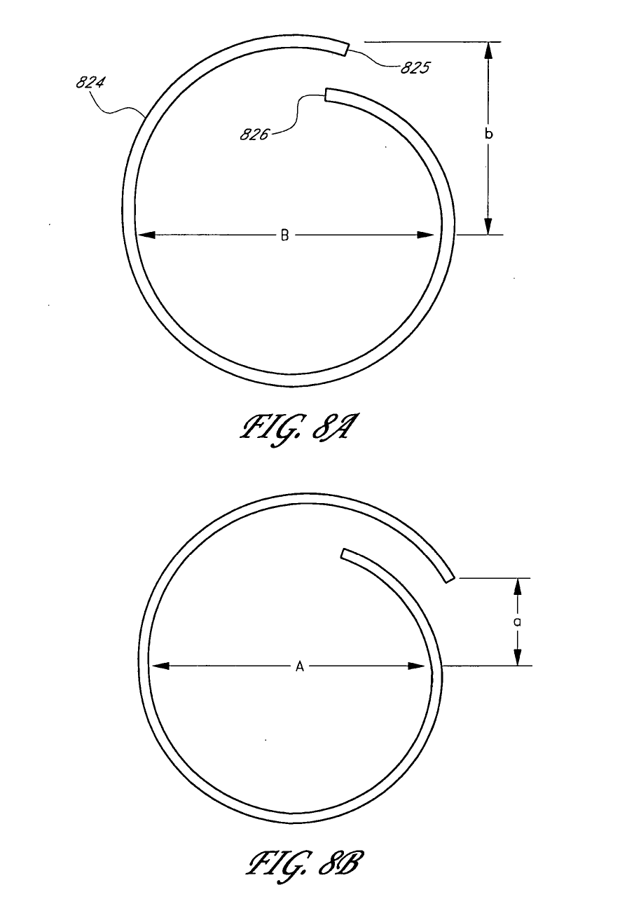

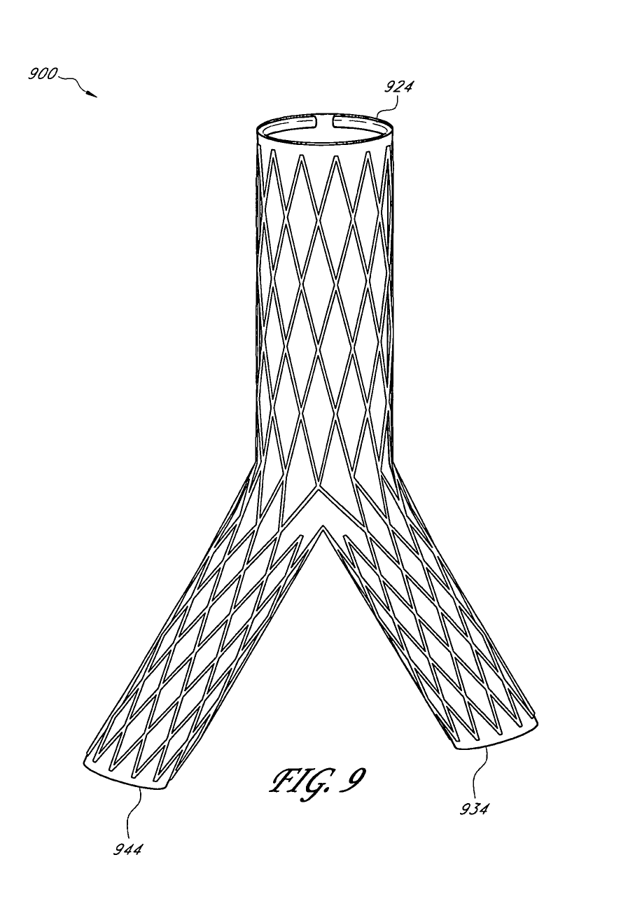

FIG. 8A illustrates an embodiment of an adjustable ring and/or adjustable element 824, which is expandable and/or contractible upon activation. The adjustable ring 824 does not form a closed shape. That is, the adjustable ring 824 comprises a first end 825 and a second end 826 that do not contact, thereby forming a C-shaped and/or G-shaped structure. In the illustrated embodiment, the adjustable ring 824 is substantially flat. In other embodiments, the adjustable ring 824 is not flat. FIG. 8B illustrates the adjustable ring 824 after activation. In the illustrated embodiment, the adjustable ring 824 contracts on activation. The dimension B in FIG. 8B is less than the corresponding dimension A FIG. 8A, and the dimension b in FIG. 8B is less than the corresponding dimension a in FIG. 8A. Those skilled in the art will understand that in other embodiments, the adjustable ring 824 expands on activation.

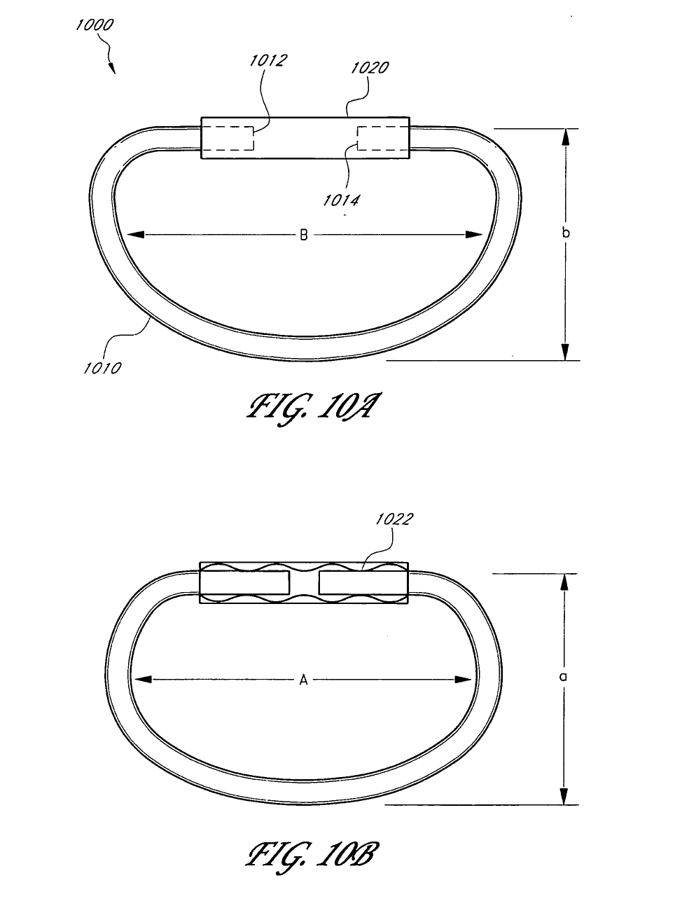

FIG. 9 illustrates an embodiment of a graft implant 900 that is similar to the embodiment illustrated in FIG. 1A, in which the aortic adjustable ring 924 is similar to the adjustable ring illustrated in FIG. 8A.

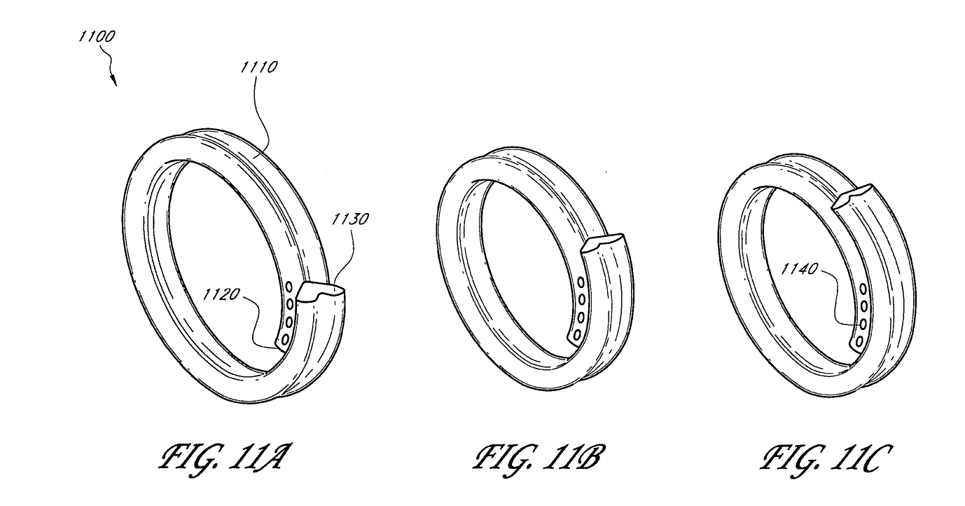

Another embodiment of an adjustable ring and/or adjustable element 1000 is illustrated in FIG. 10A comprising a ring member 1010 and a ratchet member 1020. In the illustrated embodiment, the ends of the ring member 1012 and 1014 are disposed within the ratchet member 1020. The ratchet prevents undesired size changes in the adjustable element, caused, for example, by pulsatile dilation and contraction of the aorta, common iliac arteries, and/or AAA. Suitable ratchet mechanisms are known in the art. An embodiment of the ratchet member 1020 is illustrated in cross-section in FIG. 10B. The ratchet member 1020 comprises internal gripping elements 1022 which permit one-way motion of the ends of the ring member 1012 and 1014 therein. The ring member 1010 comprises a shaped memory material, for example, nitinol. The adjustable ring 1000 is expandable and/or contractible on activation. For example, the dimensions A and a in the activated configuration (FIG. 10B) are larger than the dimensions B and b in the unactivated configuration (FIG. 10A) in some embodiments and are smaller in some embodiments. In other embodiments, one of the dimensions is larger post-activation, and the other is smaller. In still other embodiments, one of the dimensions substantially does not change on activation. In some embodiments, the entire ring member 1010 is a shape memory material, for example, nitinol, while in other embodiments, the ring member 1010 comprises a material other than a shaped memory material. For example, in some embodiments, the ring member 1010 is a composite.

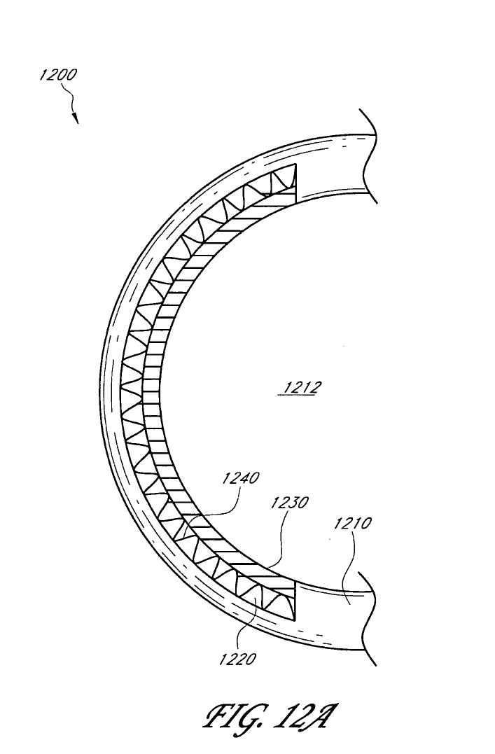

FIG. 11A illustrates another embodiment of an adjustable ring and/or adjustable element 1100 comprising a groove 1110 disposed along the outer periphery of the ring 1100. The adjustable element 1100 comprises a first end 1120, which in the illustrated embodiment, is an inner end, and a second end 1130, which in the illustrated embodiment is an outer end. In the illustrated embodiment, adjustable ring 1100 contracts upon activation as illustrated in FIGS. 11B and FIG. 11C. As illustrated in the sequence of FIGS. 11A-11C, the groove 1110 guides the first and second ends 1120 and 1130, thereby maintaining a substantially planar configuration. In other embodiments, the adjustable ring 1100 expands on activation, for example, in the sequence of FIGS. 11C-11A. Those skilled in the art will understand that in some embodiments, the groove 1110 is disposed on the inner surface of the adjustable ring 1100. FIG. 11C also illustrates holes 1140, which are useful, for example, for securing the adjustable ring 1100 to the graft implant.

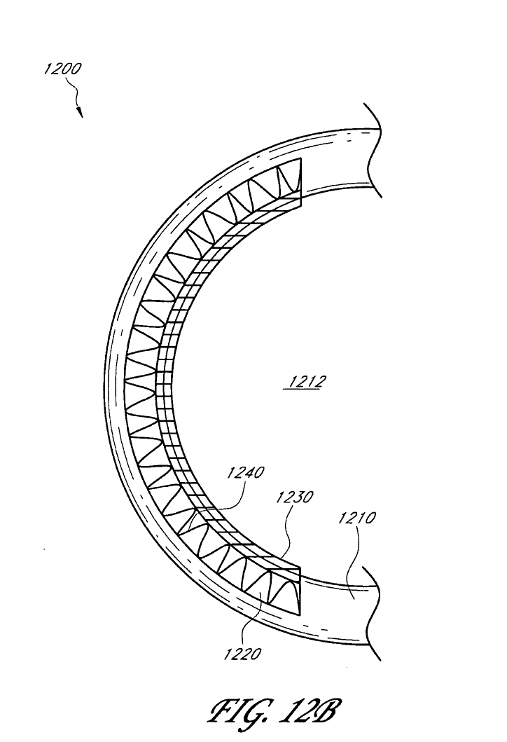

FIG. 12A illustrates in cross-section another embodiment of an adjustable element 1200 comprising a body member 1210, which comprises a recess 1220. In the illustrated embodiment, the body member 1210 is generally concave, defining a space 1212. In the illustrated embodiment, the recess 1220 is formed on the concave portion of the body member 1210. A movable member 1230 is disposed in the recess 1220. Between the body member 1210 and the movable member 1230 is disposed a shape memory element 1240. In preferred embodiments, the body member 1210 is substantially rigid, for example, a metal, a polymer resin, which is reinforced in some embodiments, or a composite. In some embodiments, the movable member 1230 is flexible, elastic, and/or elastomeric, for example, polymers, silicone rubber, synthetic rubber, fabrics, other elastomeric materials known in the art, and combinations and/or composites thereof. In other embodiments, the movable member 1230 is substantially rigid. The shape memory element 1240 comprises one or more suitable shape memory materials disclosed herein, for example, nitinol.

FIG. 12B illustrates the adjustable element 1200 after activation. In this case the shape memory element 1240 expands, thereby urging the movable member 1230 into the space 1212, thereby reducing the volume of the space 1212. Those skilled in the art will understand that, in other embodiments, the adjustable element is configured such that a movable member is disposed on a convex portion of a body member, thereby increasing the diameter of an adjustable element, while in other embodiments, the adjustable element is configured such that a movable member is disposed on a substantially planar portion of the body member, thereby increasing the length and/or width of the adjustable element.

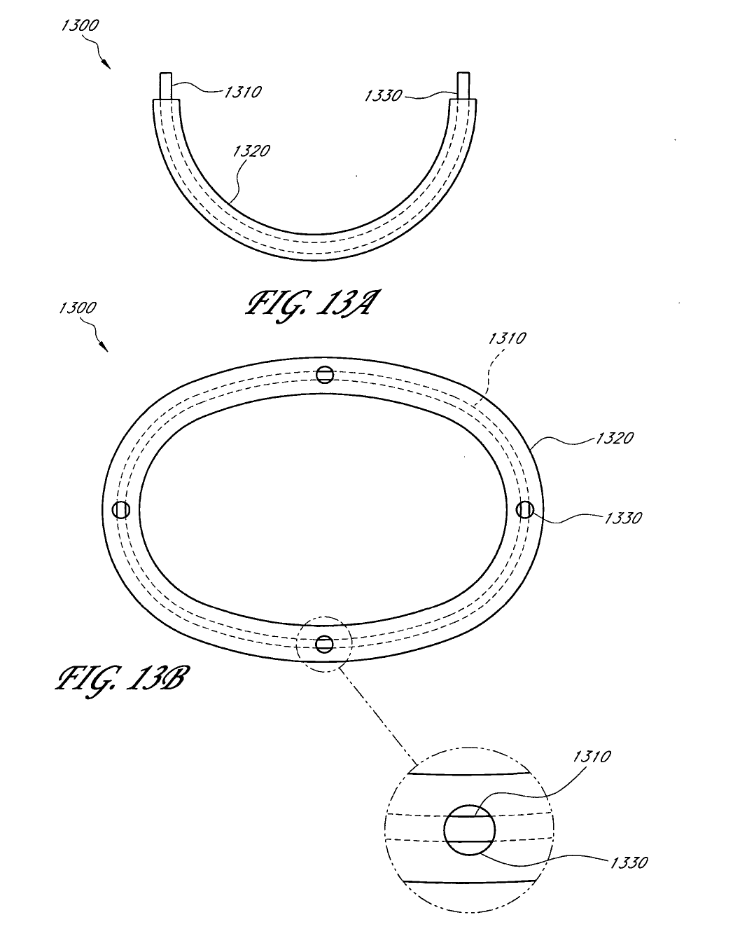

FIG. 13A illustrates an embodiment of an adjustable element 1300 comprising a U-shaped shape memory element 1310 on which is disposed a coating or layer 1320. As discussed above, suitable coatings include thermally insulators, electrical insulators, energy absorbing materials, porous materials, lubricating materials, bioactive materials, biodegradable materials, combinations thereof, and the like. In the illustrated embodiment, the layer and/or jacket 1320 is a thermal insulation layer, for example, a polymer layer. A portion of the insulating layer 1330 remains exposed in the illustrated embodiment. In some embodiments, the insulating layer 1330 also serves another function, for example, as a HIFU absorbing material, a MRI absorbing material, a lubricating layer, a drug eluting layer, a biodegradable layer, a porous layer, and combinations thereof. FIG. 13B illustrates another embodiment in which the shape memory element 1310 is a ring. A plurality of windows 1330 are provided in the insulation layer 1320. In these embodiments, the insulation layer reduces heat loss, thereby facilitating activation of the shape memory element.

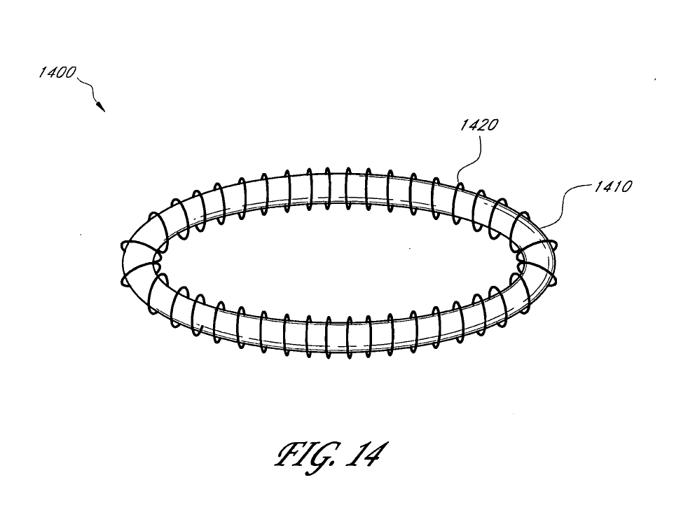

In the embodiment illustrated in FIG. 14 , an adjustable element 1400 comprises a ring-shaped shape memory element 1410 and a fine wire 1420 wrapped thereon. The fine wire 1420 is any suitable conductive wire, for example, platinum coated copper, titanium, tantalum, stainless steel, gold, and combinations thereof. As discussed above, in some preferred embodiments, the wire 1420 forms a loop suitable for inductive heating. The fine wire 1420 permits focused and/or rapid heating of the adjustable element 1400 using, for example, by induction, while reducing heating of surrounding tissue. The fine wire 1420 is from about 0.05 mm to about 0.5 mm in diameter. Those skilled in the art will understand that different wrapping geometries are also useful, for example, circumferential and/or wrapping on a bias. Some embodiments comprise additional wrapped wire, for example, in additional layers, or disposed at selected potions of the adjustable element. As discussed above, some embodiments comprise a thermally insulating, electrically insulating, protective, and/or covering layer.

In some embodiments, the adjustable elements in the graft implant are activated using one or more purpose built devices which are positioned on or around a patient's body in such a way to focus the energy on the adjustable elements. In some embodiments, the purpose built device is wrapped around the patient.

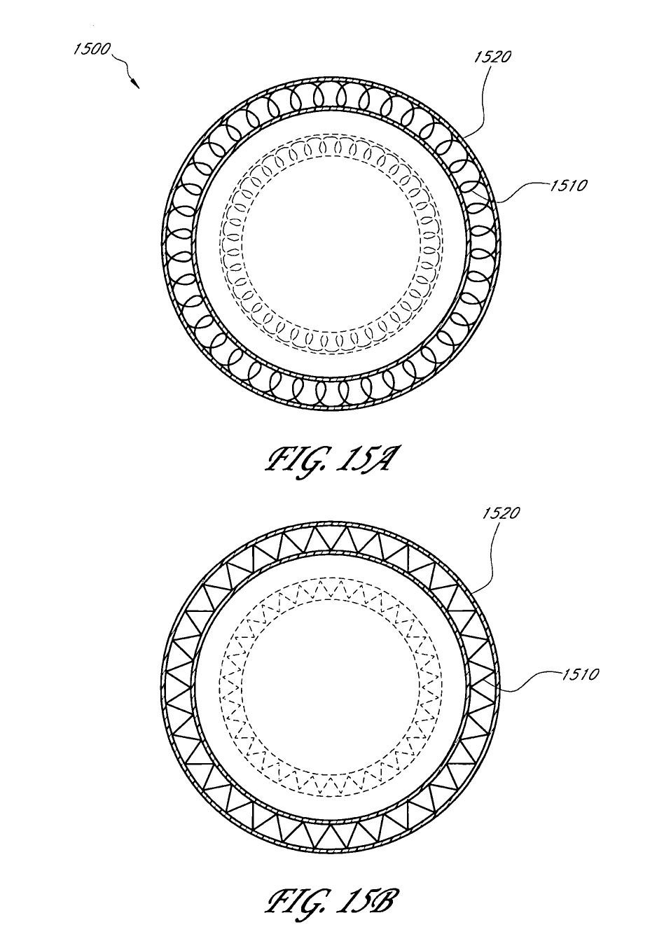

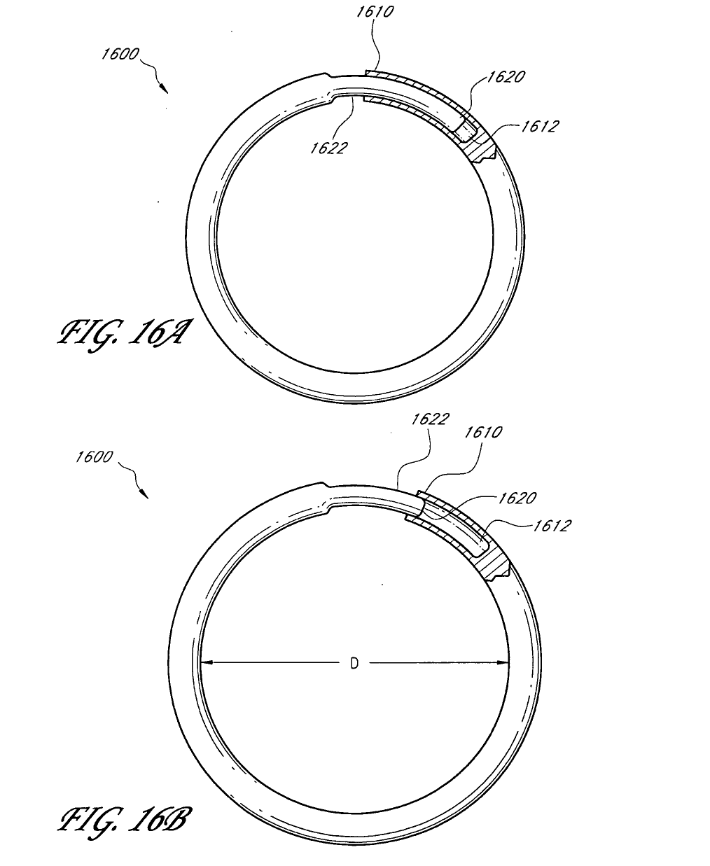

FIG. 15A and 15B illustrate cross sections of embodiments of adjustable elements 1500 comprising a convoluted shape memory element 1510 and a coating and/or layer 1520 Suitable coatings and/or layer materials are discussed above. In FIG. 15A, the convoluted shape memory element is in the shape of a coil, while in FIG. 15B, it is pleated. The unadjusted sizes of the adjustable elements 1500 are shown in phantom.

FIG. 16A illustrates in partial cross section an embodiment of a generally circular adjustable element 1600 in an unadjusted configuration comprising a first end 1610 and a second end 1620. The first end comprises a recess 1612 into which a reduced diameter portion 1622 of the second end is slidably inserted. FIG. 16B illustrates the adjustable element 1600 of FIG. 16A after adjustment, in which the reduced diameter portion 1622 is partially withdrawn from the recess 1622, and the diameter D of the adjustable element increased.

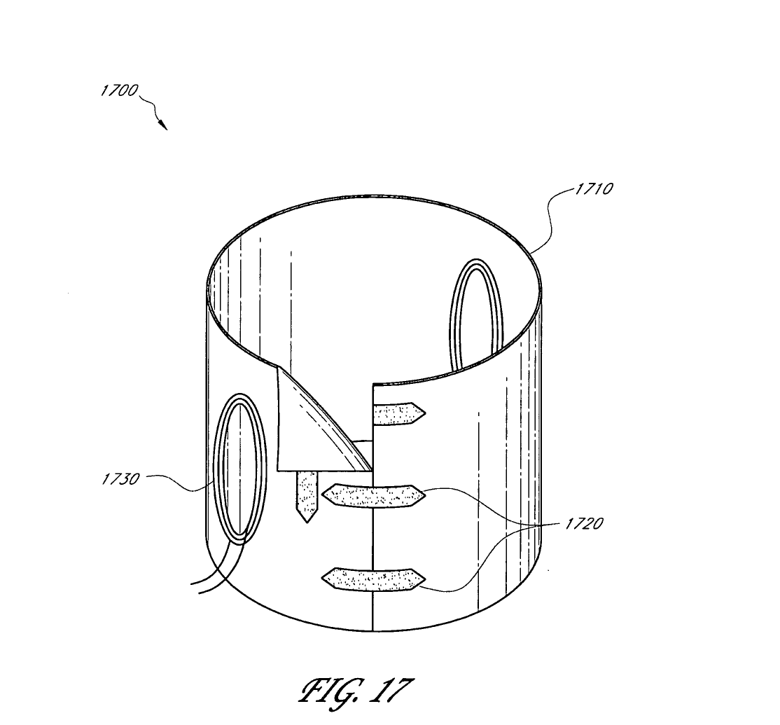

An embodiment of a wrappable inductive activation device 1700 is illustrated in FIG. 17. The device 1700 comprises a wrapping member 1710 dimensioned and configured to wrap around a patient's abdomen. The wrapping member 1700 is at least circumferentially flexible, and comprises a flexible material known in the art, for example, a woven fabric, a non-woven fabric, textile, paper, a membrane and/or film, combinations thereof and the like. In some embodiments, the wrapping member 1710 is at least the circumferentially elastic. In the illustrated embodiment, the wrapping member 1710 comprises a closure 1720, which facilitates securing and removing the device 1700 to and from a patient. Suitable closures 1720 are known in the art, for example, laces, hooks, snaps, buttons, buckles, belts, ties, slide fasteners (Zippers(®), hook and loop fasteners (Velcro®), combinations thereof, and the like. The device 1500 also comprises one or more conductive coils 1730, which are used to generate one or more electromagnetic fields for activating the graft implant. Some embodiments comprise circumferential coils.

The electrical current in the coil(s) 1730 is controlled using any suitable controller (not illustrated). In some preferred embodiments, the current control is automated, for example, using a computer, microprocessor, data processing unit, and the like. As discussed above, in some preferred embodiments, the graft implant is dynamically remodeled, that is, the graft implant contemporaneously imaged and adjusted. In some preferred embodiments, the controller is integrated with a system for imaging at least an adjustable element in the graft implant. As discussed above, in some embodiments, an adjustable element is adjusted in steps. Dynamic remodeling permits a user to monitor the effectiveness of each adjustment step.

While certain embodiments have been described, these embodiments have been presented by way of example only, and are not intended to limit the scope of the disclosure. Those skilled in the art will understand that the devices, methods, and systems described herein may be embodied in a variety of other forms. Furthermore, various omissions, substitutions and changes in the form of the devices, methods, and systems described herein may be made without departing from the teachings of this disclosure. The accompanying claims and their equivalents are intended to cover such forms or modifications.

1. An endovascular implant for treating an abdominal aortic aneurysm, the endovascular implant comprising:
a body comprising an expandable frame coupled to a graft member defining a lumen, wherein:
the body is substantially Y-shaped, defining an aortic arm, a left iliac arm, and a right iliac arm,
each arm comprises a body end and an open end, and
the open end is in fluid communication with the lumen; and
at least one adjustable element coupled to or integrated with the body and comprising a shape memory material, wherein:
the at least one adjustable element has at least a first configuration and a second configuration,
the first configuration and second configuration differ in at least one dimension, and
the at least one adjustable element is adjustable postoperatively from the first configuration to the second configuration in response to application of energy from an energy source external to a patient's body.

2. The endovascular graft implant of claim 1, wherein the shape memory material is selected from the group consisting of shape memory metals, shape memory alloys, shape memory polymers, shape memory ferromagnetic alloys, and combinations thereof.

3. The endovascular graft implant of claim 2, wherein the shape memory material comprises nitinol.

4. The endovascular graft implant of claim 1, wherein the at least one dimension of the second configuration is greater than the at least one dimension of the first configuration.

5. The endovascular graft implant of claim 4, wherein the at least one dimension is a diameter.

6. The endovascular graft implant of claim 4, wherein the at least one dimension is a length.

7. The endovascular graft implant of claim 1, wherein the at least one adjustable element is disposed in proximity to the open end of at least one of the aortic arm, the left iliac arm, and the right iliac arm.

8. The endovascular graft implant of claim 7, wherein the graft member covers at least a portion of the at least one adjustable element.

9. The endovascular graft implant of claim 7, further comprising an adjustable element disposed in proximity to the open ends of each of the other two of the aortic arm, the left iliac arm, or the right iliac arm.

10. The endovascular graft implant of claim 7, further comprising at least a second adjustable element disposed between the open end and the body end of the at least one of the aortic arm, the left iliac arm, or the right iliac arm.

11. The endovascular graft implant of claim 1, wherein the frame comprises the at least one adjustable element.

12. The endovascular graft implant of claim 11, wherein substantially the entire frame is the at least one adjustable element.

13. The endovascular graft implant of claim 1, wherein the adjustable element comprises a closed ring.

14. The endovascular graft implant of claim 13, wherein the closed ring comprises a one-way ratchet.

15. The endovascular graft implant of claim 1, wherein the adjustable element comprises an open ring.

16. The endovascular graft implant of claim 15, wherein the adjustable element comprises a spiral portion.

17. The endovascular graft implant of claim 1, wherein an insulating layer is disposed on at least a portion of the shape memory material.

18. The endovascular graft implant of claim 17, wherein portions of the shape memory material are exposed through openings in the insulating layer.

19. The endovascular graft implant of claim 1, wherein an energy-absorbing material is disposed on at least a portion of the shape memory material.

20. The endovascular graft implant of claim 19, wherein the energy absorbing material absorbs ultrasonic energy.

21. The endovascular graft implant of claim 19, wherein the energy absorbing material absorbs radio frequency energy.

22. The endovascular graft implant of claim 1, wherein a loop of wire is wrapped around at least a portion of the shape memory material.

23. An endovascular graft implant for treating an abdominal aortic aneurysm, the endovascular graft implant comprising:
means for supporting a at least a part of the endovascular graft implant;
means for causing blood flow to bypass the abdominal aortic aneurysm, the means for causing blood flow to bypass the abdominal aortic aneurysm being coupled to the means for supporting; and
means for adjusting at least a portion of the endovascular graft implant postoperatively from a first configuration to a second configuration using an energy source external to a patient's body, wherein the first configuration and second configuration differ in at least one dimension.

24. A method for treating an abdominal aortic aneurysm, the method comprising:
implanting an endovascular graft implant to cause blood flow substantially to bypass the abdominal aortic aneurysm, wherein the endovascular graft implant comprises:
a body comprising an expandable frame and a graft defining a lumen, wherein
the body is generally Y-shaped, defining an aortic arm, a left iliac arm, and a right iliac arm,
each arm comprises a body end and an open end, and
the open end is open to the lumen;
at least one adjustable element coupled to or integrated with the body, wherein
the at least one adjustable element has at least a first configuration and a second configuration,
the first configuration and second configuration differ in at least one dimension, and
the at least one adjustable element is adjustable postoperatively from the first configuration to the second configuration using an energy source external to a patient's body; and
adjusting the at least one adjustable element from the first configuration to the second configuration.

25. The method of claim 24, wherein the implanting is performed percutaneously.

26. The method of claim 25, wherein the implanting comprises expanding at least a portion of the endovascular graft implant using a balloon.

27. The method of claim 24, wherein the adjusting is performed postoperatively.

28. The method of claim 24, wherein the adjusting is performed in steps.

29. The method of claim 24, wherein the adjusting comprises applying radio frequency energy to the adjustable element.

30. The method of claim 24, wherein the adjusting comprises applying ultrasound energy to the adjustable element.

31. The method of claim 24, wherein the adjusting comprises applying magnetic energy to the adjustable element.

32. The method of claim 24, wherein the at least one adjustable element is imaged contemporaneously with the adjusting.

33. An endovascular implant for treating an aneurysm, the endovascular implant comprising:
a body comprising an expandable frame coupled to a graft member defining a lumen, wherein:
the body comprises at least a first open end and a second open end, and
the first open end and the second open end are in fluid communication with the lumen; and
at least one adjustable element coupled to or integrated with the body and comprising a shape memory material, wherein:
the at least one adjustable element has at least a first configuration and a second configuration,
the first configuration and second configuration differ in at least one dimension, and
the at least one adjustable element is adjustable postoperatively from the first configuration to the second configuration in response to application of energy from an energy source external to a patient's body.

34. The endovascular implant of claim 33, wherein the body is substantially tubular.

35. A method for treating an aneurysm, the method comprising:
implanting an endovascular graft implant to cause blood flow substantially to bypass the aneurysm, wherein the endovascular graft implant comprises:
a body comprising an expandable frame coupled to a graft member defining a lumen, wherein:
the body comprises at least a first open end and a second open end, and
the first open end and the second open end are in fluid communication with the lumen; and
at least one adjustable element coupled to or integrated with the body and comprising a shape memory material, wherein:
the at least one adjustable element has at least a first configuration and a second configuration,
the first configuration and second configuration differ in at least one dimension, and
the at least one adjustable element is adjustable postoperatively from the first configuration to the second configuration in response to application of energy from an energy source external to a patient's body; and
adjusting the at least one adjustable element from the first configuration to the second configuration.

36. The method of claim 35, wherein the body is substantially tubular.
